(19) United States
(12) Patent Application Publication
(10) Pub. No.: US 2019/0248909 A1
(43) Pub. Date: Aug. 15, 2019

(54) MODULATING AUTOIMMUNE DIABETES BY REDUCING OR REMOVING THE RESIDENT MACROPHAGES OF THE ISLETS OF LANGERHANS

(71) Applicant: Washington University, St. Louis, MO (US)

(72) Inventors: Emil R. Unanue, St. Louis, MO (US); Javier A. Carrero, St. Louis, MO (US)

(21) Appl. No.: 16/275,013

(22) Filed: Feb. 13, 2019

Related U.S. Application Data
(60) Provisional application No. 62/630,046, filed on Feb. 13, 2018.

(51) Int. Cl. C07K 16/28; A61P 3/10

(52) U.S. Cl. CPC C07K 16/2866; A61K 2039/505; C07K 2317/76; A61P 3/10

(57) ABSTRACT

The present disclosure provides methods of selectively depleting islet macrophages in the pancreas comprising disrupting the CSF-1 pathway.

### CROSS-REFERENCE TO RELATED APPLICATION

This application claims the benefit of U.S. Provisional Application No. 62/630,046, filed Feb. 13, 2018, the disclosures of which is hereby incorporated by reference in its entirety.

### GOVERNMENTAL RIGHTS

This invention was made with government support under AI014551 awarded by the National Institutes of Health. The government has certain rights in the invention.

### FIELD OF THE TECHNOLOGY

The disclosure provides methods of selectively depleting islet macrophages in the pancreas comprising targeting the CSF-1 pathway.

### BACKGROUND

Type 1 diabetes, once known as juvenile diabetes or insulin-dependent diabetes, is a chronic condition in which the pancreas produces little or no insulin, a hormone needed to allow sugar (glucose) to enter cells to produce energy. Type 1 diabetes can affect major organs in your body, including heart, blood vessels, nerves, eyes, and kidneys. The exact cause of type 1 diabetes is unknown. In most people with type 1 diabetes, the body's own immune system mistakenly destroys the insulin-producing (islet) cells in the pancreas. Despite active research, type 1 diabetes has no cure. Thus there is a need in the art for early treatment of type 1 diabetes.

### SUMMARY

In an aspect, the disclosure provides a method of depleting islet macrophages in the pancreas. The method comprises administering to a subject a composition comprising a compound that targets the CSF-1 pathway.

In another aspect, the disclosure provides a method of selectively depleting islet macrophages in the pancreas relative to tissue macrophages. The method comprises administering to a subject a composition comprising an anti-CSF1R antibody.

In still another aspect, the disclosure provides a method of delaying the onset of autoimmune diabetes. The method comprises depleting islet macrophages in the pancreas of a subject by administering to the subject a composition comprising a compound that targets the CSF-1 pathway.

### BRIEF DESCRIPTION OF THE FIGURES

The application file contains at least one drawing executed in color. Copies of this patent application publication with color drawing(s) will be provided by the Office upon request and payment of the necessary fee.

FIG. 1 depicts flow cytometry plots following αCSF-1R treatment for 3 weeks. Data shows that αCSF-1R treatment causes a 3-fold reduction in lymph node resident macrophages. Cells were gated using F4/80 and CD11b.

FIG. 2 depicts flow cytometry plots following αCSF-1R treatment for 3 weeks. Data shows that αCSF-1R treatment depletes intra-islet leukocytes. Cells were gated using SSC-A and CD45.

FIG. 3 depicts flow cytometry plots following αCSF-1R treatment for 3 weeks. Data shows that αCSF-1R treatment depletes intra-islet macrophages. Cells were gated using F4/80 and CD103.

FIG. 4 depicts flow cytometry plots following 0.25 mg, 0.5 mg or 2.0 mg αCSF-1R treatment for 2 weeks. Data shows that intra-islet macrophages remain depleted at 2 weeks after treatment with the αCSF-1R antibody. Cells were gated using SSC-A and CD45.

FIG. 5 depicts flow cytometry plots following 0.25 mg, 0.5 mg or 2.0 mg αCSF-1R treatment for 2 weeks. Data shows that intra-islet macrophages remain depleted at 2 weeks after treatment with the αCSF-1R antibody. Cells were gated using CD11c and I-Ag7.

FIG. 6 depicts flow cytometry plots following 0.25 mg, 0.5 mg or 2.0 mg αCSF-1R treatment for 2 weeks. Data shows that intra-islet macrophages remain depleted at 2 weeks after treatment with the αCSF-1R antibody. Cells were gated using F4/80 and CD103.

FIG. 7 depicts flow cytometry plots showing the timecourse of islet macrophage depletion. Cells were gated using SSC-A and CD45.

FIG. 8 depicts flow cytometry plots showing the timecourse of islet macrophage depletion. Cells were gated using CD11c and MHC II.

FIG. 9 depicts flow cytometry plots showing the timecourse of islet macrophage depletion. Cells were gated using F4/80 and CD11b.

FIG. 10 depicts flow cytometry plots showing the timecourse of islet macrophage depletion. Cells were gated using SSC-A and CD45.

FIG. 11 depicts flow cytometry plots showing the timecourse of islet macrophage depletion. Cells were gated using CD11c and I-NE.

FIG. 12 depicts flow cytometry plots showing the timecourse of islet macrophage depletion. Cells were gated using F4/80 and CD11b.

FIG. 13 depicts flow cytometry plots showing the timecourse of stromal macrophage depletion. Cells were gated using F4/80 and CD11b.

FIG. 14 depicts flow cytometry plots showing the timecourse of lung macrophage depletion. Cells were gated using F4/80 and CD11b.

FIG. 15 depicts flow cytometry plots showing the timecourse of splenic macrophage depletion. Cells were gated using F4/80 and CD11b.

FIG. 16 depicts flow cytometry plots showing the timecourse of pLN macrophage depletion. Cells were gated using F4/80 and CD11b.

FIG. 17A, FIG. 17B, and FIG. 17C depict blood glucose measurements at 2 weeks (FIG. 17A), 3 weeks (FIG. 17B), and 4 weeks (FIG. 17C).

FIG. 18A, FIG. 18B, FIG. 18C, and FIG. 18D depict a summary of islet infiltration at 6 an d10 weeks in αCSF-1R treated NOD mice. (FIG. 18A) CD45⁺ infiltrate, (FIG. 18B) CD3ε⁺, (FIG. 18C) F4/80⁺ macrophage, and (FIG. 18D) CD103⁺ DC.

FIG. 19 depicts flow cytometry plots showing αCSF-1R treatment at 2 weeks, 3 weeks, and 6 weeks in 8 week old NOD mice. Cells were gated using SSC-A and CD45.

FIG. 20 depicts flow cytometry plots showing αCSF-1R treatment at 2 weeks, 3 weeks, and 6 weeks in 8 week old NOD mice. Cells were gated using CD11c and I-Ag⁷.

FIG. 21 depicts flow cytometry plots showing αCSF-1R treatment at 2 weeks, 3 weeks, and 6 weeks in 8 week old NOD mice. Cells were gated using F4/80 and CD103.

FIG. 22 depicts flow cytometry plots showing αCSF-1R treatment at 2 weeks, 3 weeks, and 6 weeks in 8 week old NOD mice. Cells were gated using CD3 and CD8.

FIG. 23A and FIG. 23B depict graphs showing αCSF-1R treatment at 2 weeks, 3 weeks, and 6 weeks in 8 week old NOD mice. Graphs represent percentage of CD45⁺ cells (FIG. 23A) and percentage of cells in islet (FIG. 23B).

FIG. 24A, FIG. 24B, FIG. 24C, FIG. 24D, FIG. 24E, and FIG. 24F illustrates that treatment of C57BL/6 mice with AFS98 antibody depleted their macrophages. (FIG. 24A) Female B6 mice aged 6-8 weeks were administered 0.25, 0.50, or 2.0 mg of AFS98 antibody i.p. Islets were examined 7 and 14 days after injection for the presence of macrophages. Box indicates CD45⁺ CD11c⁺MHCII⁺F4/80⁺ CD11b⁺ cells as a percent of total islet cellularity. (FIG. 24B) Female B6 mice aged 6-8 weeks were treated with 2.0 mg of AFS98, and their islets were examined at the time points indicated for the presence of macrophages. Top show the CD45⁺ cells, and Bottom show the CD45⁺ CD11c⁺MHCII⁺ F4/80⁺ CD11b⁺ cells as a percent of total islet cellularity. (FIG. 24C) Graph of the CD45⁺ cells found in mice 1-2 weeks after treatment with 2.0 mg of AFS98. (FIG. 24D) Graph of CD45⁺F4/80⁺ CD64⁺ islet stromal macrophage populations as a percent of CD45⁺ cells in control and AFS98-treated mice 2 weeks after treatment. (FIG. 24E) Mice were treated for 1-2 weeks with AFS98 antibody, and the percent of total macrophages in lung, liver, spleen, and pancreatic lymph nodes were determined. (FIG. 24F) Macrophages were examined as in FIG. 24E except plots show the percent of the indicated subpopulation of macrophages. For all graphs, controls were untreated age-matched mice. Flow cytometry plots in FIG. 24A and FIG. 24B are representative of individual islet, lung, liver, spleen, and lymph nodes, from two to three experiments with two to four mice per treatment group. Scatter plots in FIG. 24E and FIG. 24F were calculated from three independent experiments with two to three mice per group. P values were calculated using Mann-Whitney U test with the following style: not significant (n.s., >0.1234), *P=0.0332, **P=0.0021, ***P=0.0002, ****P<0.0001.

FIG. 25 illustrates that pancreatic stromal macrophages are reduced following AFS98 treatment. B6 mice were treated as in FIG. 24A, FIG. 24B, FIG. 24C, FIG. 24D, FIG. 24E, and FIG. 24F and analyzed for the presence of CD45′F4/80′CD64′ macrophages. Numbers indicate the percentage of macrophages in the pancreatic stroma as a percent of the CD45′ cells. Cells were gated on FSC/SSC/CD45′. Data are taken from two independent mice per group and is representative of two independent experiments.

FIG. 26 illustrates that thymic T cells and bone marrow B cells are grossly normal following AFS98 treatment. Male NOD mice were injected with 2 mg of either AFS98 or Rat IgG2ₐ at 4 weeks of age and then sampled at 6 weeks of age. Thymus and bone marrow were harvested, and single-cell suspensions were generated. Cells were evaluated by flow cytometry. For thymus, cells were first gated on forward and side scatter, and CD45⁺ then CD4 by CD8 was plotted. Next, the CD4/CD8 double negative population was gated and examined for CD44 and CD25 as plotted on the Right. For bone marrow, cells were gated by forward and side scatter, and then plotted for B220 expression. The B220 intermediate cells were then gated and plotted for CD24 and Ly51 staining as shown on the Right. Numbers indicate the percentage of cells in each region as a function of the total cells in the plot. Results are representative of three individual mice per group

FIG. 27A, FIG. 27B, and FIG. 27C depict islet function after depletion of macrophages by AFS98. (FIG. 27A) B6 mice were given 2.0 mg of AFS98 i.p. at 6-8 weeks of age. Glucose tolerance assays were then performed on AFS98-treated and untreated mice. After the indicated number of days, the mice were fasted for 12 hours and then injected with 2.0 g/kg glucose i.p. Blood glucose (mg/dL) was measured at the indicated time points. Results are pooled from two independent experiments (n=2-3 mice per group). (FIG. 27B) Nine-week-old B6 females were left untreated or administered 2.0 mg of AFS98 i.p. Four days later, the mice were placed on a 20% sucrose diet for an additional 7 days, then returned to a normal diet for 2 days. The mice were then killed, and the insulin content of their islets was measured (n=5 mice per group). (FIG. 27C) Three-week-old C57BL/6 mice were left untreated or administered 2.0 mg of AFS98 i.p. At 6 weeks of age, their islets were isolated, total RNA was extracted, and transcripts were analyzed by microarray. Scatter plot shows the log₂ mean expression values for four control and experimental mice. The dots highlighted in blue represent genes differentially expressed between treated and control mice at 99% confidence using moderated t test with Benjamini-Hochberg false discovery rate analysis. The selected genes are plotted in the heat map using Euclidean distance and normalized global expression as indicated.

FIG. 28A and FIG. 28B illustrate the effect of AFS98 treatment on NOD mice. (FIG. 28A) Male NOD mice at 4-5 weeks of age were administered 0.25, 0.50, or 2.0 mg of AFS98 i.p., and their islets were examined by flow cytometry. Plots show the CD45⁺ CD11c⁺MHCII⁺ gate at either 7 or 14 days after treatment. Results are representative of two experiments performed in duplicate. Values in the box represent the percent of cells as a function of total islet cellularity. (FIG. 28B) Two-week-old male NOD mice were left untreated (Control) or injected i.p. with 0.5 mg of AFS98 at 2 weeks of age and 2.0 mg of AFS98 at 4 weeks of age (AFS98). At 6 weeks of age, the islets were harvested, dispersed, and tested for their MHCII-peptide presentation to two T cell hybridomas that recognize insulin. High (25.0 mM) or low (5.0 mM) glucose and two different insulin peptides (Ins B:12-20 and Ins B:13-21) were evaluated. Bars represent the mean±SD of ³H incorporation by the IL-2-dependent cell line CTLL-2.

FIG. 29A and FIG. 29B illustrate that AFS98 depletes islet macrophages in the NOD.Rag1^{-/-} mouse. Female NOD.Rag1^{-/-} mice were treated at 4 weeks of age with 2 mg of either rat IgG2a or ASF98 and then sampled at 6 weeks of age. (FIG. 29A and FIG. 29B) Islet leukocytes cells were analyzed by flow cytometry. Cells were gated by forward and side scatter and then plotted for CD45 (Left). Next, cells were gated on CD45⁺ CD11c⁺ I-Ag7⁺, and the F4/80 by CD103 cells were plotted (Right). Values represent the percent of each box as a function of total islet cellularity. (FIG. 29B) Scatter plots of the percent of CD45⁺ or F4/80⁺ CD45⁺ CD11c⁺ I-Ag7⁺ in two individual mice per group.

FIG. 30A and FIG. 30B illustrate that depletion of macrophages blocks MHC-II antigen presentation by whole islet cells. Antigen presentation was performed as in FIG. 29B except with 6-weeks-old B6.g7 mice that were given 2 mg of either control or AFS98 antibody at 4 weeks of age. FIG. 30A shows the CTLL-2 assay for an anti-insulin CD4 T cell hybridoma. FIG. 30B shows the peptide curve of the hybridoma using an immortalized cell line as APC.

FIG. 31A, FIG. 31B, FIGS. 31C, and 31D illustrate that AFS98 treatment does not affect T cell division in lymph nodes but prevents T cell entry into islets of Langerhans. NOD mice were injected with AFS98 antibody at a dose of 0.5 mg at 2 weeks of age and 2.0 mg at 4 weeks of age. Two TCR transgenic T cells, the CD4⁺ BDC2.5 and the CD8⁺ NY8.3, were isolated from lymph nodes and spleens of their respective mice. T cells were then labeled with CFSE and transferred into 6-week-old NOD mice that had either been left untreated or treated with AFS98. (FIG. 31A and FIG. 31B) Seven days after T cell transfer, the pancreatic and inguinal lymph nodes were isolated and analyzed by flow cytometry. (FIG. 31A) Dilution of CFSE in either inguinal (Upper) or pancreatic (Lower) lymph nodes for an individual mouse per treatment is shown. Cells were gated on forward and side-scatter, CD45, CD3, and either CD4 (BDC2.5) or CD8 (NY.8.3). (FIG. 31B) Summary of division index and proliferation index for individual mice examined as in FIG. 31A. Results show three or four individual mice per group. (FIG. 31C) Ten days after TCR transgenic T cell transfer, islets of Langerhans were isolated and examined for entry of either the BDC2.5 or NY8.3 T cells by flow cytometry. The Left two images are gated on forward and side-scatter, CD45, and CD3. The Right images are gated on forward and side-scatter. BDC T cells were identified using a clonotypic antibody to its T cell receptor. NY8.3 T cells were identified by a CD45.2 congenic label (FIG. 31D). Numbers indicate the percent of cells in each selection as a function of CD45⁺ cells. T cell islet entry results are representative of two to three independent experiments with two to four individual mice per group.

FIG. 32A and FIG. 32B illustrate that Lymph node priming is not reduced by AFS98 treatment. B6.g7 or NOD mice were treated with 2 mg of AFS98 antibody for 1 week and then injected with 10 nmols INS:9-23, HEL, or IGRP peptides in complete Freund's adjuvant. The draining popliteal lymph nodes were isolated and tested by ELISPOT for IL-2 (FIG. 32A) and IFN-γ (FIG. 32B) production. Recall antigens for the ELISPOT are shown in the figure and include the following: insulin protein (INS) and INS:9-23, HEL protein and peptides that elicit CD4 (11-25) and CD8 (20-35) responses, and the IGRP peptides that elicit CD4 (128-142) and CD8 (206-214) responses. Results are taken from two individual mice tested in duplicate or triplicate.

FIG. 33A, FIG. 33B, and FIG. 33C illustrate that treatment with AFS98 protects against autoimmune diabetes. (FIG. 33A) Female NOD mice were left untreated or injected with Rat IgG2ₐ or AFS98 at the ages and doses indicated in FIG. 33C and followed for diabetes incidence. Control mice represent the pooled results of the three experiments. (FIG. 33B) Splenocytes were isolated from nondiabetic 44- to 50-week-old mice taken from FIG. 33A, 10⁷ were transferred into NOD.Rag1^{-/-} mice, and recipients were followed for diabetes incidence. (FIG. 33B) Summary of primary and transfer diabetes incidence for three AFS98 treatment protocols as well as the effect of PD-1 treatment on the AFS98 protection. Weeks indicate ages of mice at treatment, and dose is milligrams per mouse for either control or AFS98 antibodies.

FIG. 34A, FIG. 34B, FIG. 34C, FIG. 34D, FIG. 34E, and FIG. 34F illustrate that early treatment with AFS98 reduces infiltrating leukocytes in NOD mice up to 12 weeks of age. (FIG. 34A and FIG. 34B) Female NOD mice were left untreated or injected with 0.5 mg of AFS98 i.p. at 2 weeks of age and 2.0 mg of AFS98 i.p. at 4, 7, and 10 weeks of age. The islets of mice at 6 (FIG. 34B), 8 (FIG. 34A and FIG. 34B), and 12 (FIG. 34A) weeks of age were isolated and analyzed by flow cytometry. (FIG. 34A) Shows the flow cytometry plots of myeloid and T cell compartments. Gating is indicated over the plots. Flow cytometry plots are representative of individual islet preparation from three control or four AFS98-treated mice. (FIG. 34B) Summarizes the flow cytometry data for all time course experiments (three to four mice per group). Bars represent the mean±SD for two independent experiments with two to three replicates per group. (FIG. 34C) Flow cytometry plots of immune cell populations in islets isolated from nondiabetic control or AFS98-treated mice taken from FIG. 33A and FIG. 33C early treatment examined at 40-44 weeks of age. Plots were generated from individual mice. Gates are indicated on the Top of each column of plots. Flow cytometry plots are representative of individual islets preparations isolated from two control and eight AFS98-treated mice. (FIG. 34D) Summary of the data shown in FIG. 34C. (FIG. 34E and FIG. 34F) Hematoxylin/eosin staining of pancreatic sections isolated from nondiabetic AFS98-treated mice taken from FIG. 33A: The mice were treated early, and their islets were examined at 40-44 weeks of age. (Scale bars, 400 μm.)

FIG. 35 illustrates that leukocyte infiltrates are reduced following macrophage depletion at 10 weeks of age. NOD mice were either treated with AFS98 or control Rat IgG2ₐ starting at 10 weeks of age. At 22 or 40 weeks of age, the islet cells of control or AFS-treated mice were isolated and analyzed by flow cytometry as indicated. Values in the CD45 by SSC plot represent the percent of leukocytes in islets. Values in the CD3e by I-A^{g7} plot represent the percent CD3e⁺ or I-A^{g7+} cells as a percent of CD45⁺ cells. Values in the T cell (CD3e⁺, I—Ag⁷⁻) and APC (CD3e⁻, I—Ag⁷⁺) plots represent the percent of cells in each quadrant as a function of total islet cellularity. Gating is indicated over the top for each column of plots. Results are representative of two mice per group.

FIG. 36A and FIG. 36B depict graphs showing percent of leukocytes (total cells, FIG. 36A) or macrophages in islets (CD45+ cells, FIG. 36B). FIG. 36A is a composite of three independent experiments. FIG. 36B is one representative experiment.

### DETAILED DESCRIPTION

Provided herein are methods depleting or selectively depleting islet macrophages in the pancreas. Further provided are methods for delaying the onset of autoimmune diabetes in a subject. The Applicants have found macrophage depletion, either at the start of the autoimmune process or when diabetogenesis is active, leads to a significant reduction in diabetes incidence. By depleting islet macrophages the entrance of T cells into islets is reduced and results in the absence of antigen presentation. Suitable compositions and methods are detailed below.

### (I) Composition

One aspect of the present disclosure encompasses a composition comprising a compound that disrupts the colony-stimulating factor 1 (CSF-1) pathway. As used herein, the CSF-1 pathway includes the CSF-1 receptor (CSF-1R)(NCBI Reference Sequence: NP_001275634.1) and any ligand that interacts with the receptor, such as CSF-1 (M-CSF) or IL-34. The CSF-1R may also be referred to as the M-CSF receptor or CD115. The protein encoded by CSF1R gene is the receptor for colony stimulating factor 1, a cytokine which controls the production, differentiation, and function of macrophages. This receptor mediates most if not all of the biological effects of this cytokine. Ligand binding activates the receptor kinase through a process of oligomerization and transphosphorylation. The encoded protein is a tyrosine kinase transmembrane receptor and member of the CSF1/PDGF receptor family of tyrosine-protein kinases. Mutations in this gene have been associated with a predisposition to myeloid malignancy. The first intron of this gene contains a transcriptionally inactive ribosomal protein L7 processed pseudogene oriented in the opposite direction. Alternative splicing results in multiple transcript variants. Expression of a splice variant from an LTR promoter has been found in Hodgkin lymphoma (HL), HL cell lines and anaplastic large cell lymphoma.

A composition of the invention may optionally comprise one or more additional drugs or therapeutically active agents in addition to a compound that disrupts the colony-stimulating factor 1 (CSF-1) pathway. A composition of the invention may further comprise a pharmaceutically acceptable excipient, carrier or diluent. Further, a composition of the invention may contain preserving agents, solubilizing agents, stabilizing agents, wetting agents, emulsifiers, sweeteners, colorants, odorants, salts (substances of the present invention may themselves be provided in the form of a pharmaceutically acceptable salt), buffers, coating agents or antioxidants.

(a) A Compound that Disrupts the CSF-1 Pathway

A compound that disrupts the CSF-1 pathway may disrupt the pathway by specifically binding to a pathway member, may disrupt the pathway by inhibiting interaction between a pathway member and CSF-1R, or may otherwise alter signaling through the CSF-1R. In some embodiments, the altered signaling through the CSF-1R is a downregulation of activation of the receptor compared to a CSF-1R in the absence of the compound that disrupts the CSF-1 pathway. The downregulation may be in the presence or absence of a CSF-1R agonist. In certain embodiments, a compound of the invention specifically binds to CSF-1, IL-34, or CSF-1R. In particular embodiments, the compound specifically binds CSF-1R. In other embodiments, the compound interferes with the interaction between CSF-1 and CSF-1R. In each of the above embodiments, the CSF-1 pathway is disrupted in a mammal. In particular embodiments, the CSF-1 pathway is disrupted in humans or non-human primates. In other embodiments, the CSF-1 pathway is disrupted in rodents.

### (i) Antibodies

In some embodiments, the composition comprises an antibody that that disrupts the CSF-1 pathway. The term “antibody” encompasses the various forms of antibodies including but not being limited to whole antibodies, antibody fragments, humanized antibodies, chimeric antibodies, T cell epitope depleted antibodies, and further genetically engineered antibodies as long as the characteristic properties according to the invention are retained.

“Antibody fragments” comprise a portion of a full length antibody, preferably the variable domain thereof, or at least the antigen binding site thereof. Examples of antibody fragments include diabodies, single-chain antibody molecules, and multispecific antibodies formed from antibody fragments. scFv antibodies are, e.g., described in Houston, J. S., Methods in Enzymol. 203 (1991) 46-88). In addition, antibody fragments comprise single chain polypeptides having the characteristics of a VH domain binding to, for example, CSF-1, IL-34, or CSF-1R, namely being able to assemble together with a VL domain, or of a VL domain binding to, for example, CSF-1, IL-34, or CSF-1R, namely being able to assemble together with a VH domain to a functional antigen binding site and thereby providing the property.

The terms “monoclonal antibody” or “monoclonal antibody composition” as used herein refer to a preparation of antibody molecules of a single amino acid composition.

The term “chimeric antibody” refers to a monoclonal antibody comprising a variable region, i.e., binding region, from mouse and at least a portion of a constant region derived from a different source or species, usually prepared by recombinant DNA techniques. Chimeric antibodies comprising a mouse variable region and a human constant region are especially preferred. Such rat/human chimeric antibodies are the product of expressed immunoglobulin genes comprising DNA segments encoding rat immunoglobulin variable regions and DNA segments encoding human immunoglobulin constant regions. Other forms of “chimeric antibodies” encompassed by the present invention are those in which the class or subclass has been modified or changed from that of the original antibody. Such “chimeric” antibodies are also referred to as “class-switched antibodies.” Methods for producing chimeric antibodies involve conventional recombinant DNA and gene transfection techniques now well known in the art. See, e.g., Morrison, S. L., et al., Proc. Natl. Acad Sci. USA 81 (1984) 6851-6855; U.S. Pat. Nos. 5,202,238 and 5,204,244.

The term “CDR-grafted variant” as used within the current application denotes a variable domain of an antibody comprising complementary determining regions (CDRs or hypervariable regions) from one source or species and framework regions (FRs) from a different source or species, usually prepared by recombinant DNA techniques. CDR-grafted variants of variable domains comprising murine CDRs and a human FRs are preferred.

The term “T-cell epitope depleted variant” as used within the current application denotes a variable domain of an antibody which was modified to remove or reduce immunogenicity by removing human T-cell epitopes (peptide sequences within the variable domains with the capacity to bind to MHC Class II molecules). By this method interactions between amino acid side chains of the variable domain and specific binding pockets with the MHC class II binding groove are identified. The identified immunogenic regions are mutated to eliminate immunogenicity. Such methods are described in general in, e.g., WO 98/52976.

The term “humanized variant” as used within the current application denotes a variable domain of an antibody, which is reconstituted from the complementarity determining regions (CDRs) of non-human origin. e.g. from a non-human species, and from the framework regions (FRs) of human origin, and which has been further modified in order to also reconstitute or improve the binding affinity and specificity of the original non-human variable domain. Such humanized variants are usually prepared by recombinant DNA techniques. The reconstitution of the affinity and specificity of the parent non-human variable domain is the critical step, for which different methods are currently used. In one method it is determined whether it is beneficial to introduce mutations, so called backmutations, in the non-human CDRs as well as in the human FRs. The suited positions for such backmutations can be identified e.g. by sequence or homology analysis, by choosing the human framework (fixed frameworks approach; homology matching or best-fit), by using consensus sequences, by selecting FRs from several different human mAbs, or by replacing non-human residues on the three dimensional surface with the most common residues found in human mAbs (“resurfacing” or “veneering”).

The “variable domain” (variable domain of a light chain (VL), variable domain of a heavy chain (VH)) as used herein denotes each of the pair of light and heavy chain domains which are involved directly in binding the antibody to the antigen. The variable light and heavy chain domains have the same general structure and each domain comprises four framework (FR) regions whose sequences are widely conserved, connected by three “hypervariable regions” (or complementary determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody's heavy and light chain CDR3 regions play a particularly important role in the binding specificity/affinity of the antibodies according to the invention and therefore provide a further object of the invention.

The term “antigen-binding portion of an antibody” when used herein refer to the amino acid residues of an antibody which are responsible for antigen-binding. The antigen-binding portion of an antibody comprises amino acid residues from the “complementary determining regions” or “CDRs”. “Framework” or “FR” regions are those variable domain regions other than the hypervariable region residues as herein defined. Therefore, the light and heavy chain variable domains of an antibody comprise from N- to C-terminus the domains FR1, CDR1, FR2, CDR2, FR3, CDR3, and FR4. Especially. CDR3 of the heavy chain is the region which contributes most to antigen binding and defines the antibody's properties. CDR and FR regions are determined according to the standard definition of Kabat et al., Sequences of Proteins of Immunological Interest, 5th ed., Public Health Service, National Institutes of Health, Bethesda, Md. (1991) and/or those residues from a “hypervariable loop”.

In some embodiments, the antibody may be an anti-CSF-1 antibody, anti-IL-34 antibody, or anti-CSF-1R antibody. In an exemplary embodiment, the antibody may be an anti-CSF-1R antibody. Non-limiting examples of suitable antibodies may include AFS-98.

### (ii) Small Molecule Tyrosine Kinase Inhibitors

In some embodiments, the composition comprises a small molecule tyrosine kinase inhibitor that disrupts the CSF-1 pathway. In this manner, CSF-1R is a tyrosine kinase transmembrane receptor and member of the CSF1/PDGF receptor family of tyrosine-protein kinases. Hence, signaling through CSF-1R may be disrupted by tyrosine kinase inhibitors. Non-limiting examples of suitable small molecule tyrosine kinase inhibitors may include, without limit, JNJ-28312141 (see Mol Cancer Ther. 2009 November; 8(11):3151-61, incorporated herein by reference), JNJ-44679292, JNJB:44679292, JNJ-40346527. In some embodiments, the CSF1R inhibitor is DCC-3014.

### b) Components of the Composition

The present disclosure also provides pharmaceutical compositions. The pharmaceutical composition comprises a compound that disrupts the CSF-1 pathway, as an active ingredient, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

In each of the embodiments described herein, a composition of the invention may optionally comprise one or more additional drug or therapeutically active agent in addition to a compound that disrupts the CSF-1 pathway. Thus, in addition to the therapies described herein, one may also provide to the subject other therapies known to be efficacious for treatment of the disease, disorder, or condition. In some embodiments, the additional drug or therapeutically active agent induces anti-inflammatory effects. In some embodiments, the secondary agent is an antibody. In some embodiments, the secondary agent is selected from a corticosteroid, a non-steroidal anti-inflammatory drug (NSAID), an intravenous immunoglobulin, a tyrosine kinase inhibitor, a fusion protein, a monoclonal antibody directed against one or more pro-inflammatory cytokines, or a combination thereof. In some embodiments, the secondary agent may be a glucocorticoid, a corticosteroid, a non-steroidal anti-inflammatory drug (NSAID), or a phenolic antioxidant. In some embodiments, the secondary agent is an anti-inflammatory drug. In some embodiments, anti-inflammatory drugs include, but are not limited to, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, curcumin, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, lysofylline, meclofenamate sodium, meclofenamic acid, meclorisone dibutyrate, mefenamic acid, mesalamine, meseclazone, methylprednisolone suleptanate, mom iflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, mepolizumab, prodrugs thereof, and a combination thereof.

(i) Diluent

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

(ii) Binder

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

(iii) Filler

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

(iv) Buffering Agent

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

(v) pH Modifier

In various embodiments, the excipient may be a pH modifier. By way of non-limiting example, the pH modifying agent may be sodium carbonate, sodium bicarbonate, sodium citrate, citric acid, or phosphoric acid.

(vi) Disintegrant

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

(vii) Dispersant

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

(viii) Excipient

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

(ix) Lubricant

In a further embodiment, the excipient may be a lubricant. Non-limiting examples of suitable lubricants include minerals such as talc or silica; and fats such as vegetable stearin, magnesium stearate, or stearic acid.

(x) Taste-Masking Agent

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

(xi) Flavoring Agent

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

(xii) Coloring Agent

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

### (c) Administration

(I) Dosage Forms

The composition can be formulated into various dosage forms and administered by a number of different means that will deliver a therapeutically effective amount of the active ingredient. Such compositions can be administered orally (e.g. inhalation), parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, intra-articular, or intrasternal injection, or infusion techniques. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980). In a specific embodiment, a composition may be a food supplement or a composition may be a cosmetic.

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof. For administration by inhalation, the compounds are delivered in the form of an aerosol spray from pressured container or dispenser which contains a suitable propellant, e.g., a gas such as carbon dioxide, or a nebulizer.

For parenteral administration (including subcutaneous, intradermal, intravenous, intramuscular, intra-articular and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as ethylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules, and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols, or oils. In some embodiments, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles, and mouth washes. Transmucosal administration may be accomplished through the use of nasal sprays, aerosol sprays, tablets, or suppositories, and transdermal administration may be via ointments, salves, gels, patches, or creams as generally known in the art.

In certain embodiments, a composition comprising a compound that disrupts the CSF-1 pathway, is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present invention. Non-limiting examples of suitable structured fluid delivery systems may include nanoparticles, liposomes, microemulsions, micelles, dendrimers, and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery of a compound that disrupts the CSF-1 pathway, in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, a compound that disrupts the CSF-1 pathway may be selectively delivered to a cell by encapsulation in a liposome that fuses with the targeted cell's membrane.

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9,12,15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying a compound that disrupts the CSF-1 pathway, may be prepared by any known method of preparing liposomes for drug delivery, such as, for example, detailed in U.S. Pat. Nos. 4,241,046; 4,394,448; 4,529,561; 4,755,388; 4,828,837; 4,925,661; 4,954,345; 4,957,735; 5,043,164; 5,064,655; 5,077,211; and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of the itaconate, malonate, derivatives thereof, a compound that disrupts the CSF-1 pathway, concentration and composition of lipid, concentration of multivalent cations, rate of mixing, presence of and concentration of solvent.

In another embodiment, a composition of the invention may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. A compound that disrupts the CSF-1 pathway may be encapsulated in a microemulsion by any method generally known in the art.

In yet another embodiment, a compound that disrupts the CSF-1 pathway, may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

Generally, a safe and effective amount of a compound that disrupts the CSF-1 pathway is, for example, that amount that would cause the desired therapeutic effect in a subject while minimizing undesired side effects. In various embodiments, an effective amount of a compound that disrupts the CSF-1 pathway described herein can substantially reduce CSF-1R activation, selectively depletes islet macrophages in the pancreas relative to tissue macrophages, slows the progress of autoimmune diabetes, or delays the onset of autoimmune diabetes.

When used in the treatments described herein, a therapeutically effective amount of a compound that disrupts the CSF-1 pathway can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to substantially reduce CSF-1R activation, selectively deplete islet macrophages in the pancreas, slow the progress of autoimmune diabetes, or delay the onset of autoimmune diabetes.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a compound that disrupts the CSF-1 pathway can occur as a single event or over a time course of treatment. For example, a compound that disrupts the CSF-1 pathway can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for autoimmune diabetes or inflammatory disease.

A compound that disrupts the CSF-1 pathway can be administered simultaneously or sequentially with another agent, such as an antibiotic, an anti-inflammatory, or another agent. For example, a compound that disrupts the CSF-1 pathway can be administered simultaneously with another agent, such as an antibiotic or an anti-inflammatory. Simultaneous administration can occur through administration of separate compositions, each containing one or more of a compound that disrupts the CSF-1 pathway, an antibiotic, an anti-inflammatory, or another agent. Simultaneous administration can occur through administration of one composition containing two or more a compound that disrupts the CSF-1 pathway, an antibiotic, or an anti-inflammatory. A compound that disrupts the CSF-1 pathway can be administered sequentially with an antibiotic, an anti-inflammatory, or another agent. For example, a compound that disrupts the CSF-1 pathway can be administered before or after administration of an antibiotic, an anti-inflammatory, or another agent.

### (II) Methods

The present disclosure encompasses a method depleting islet macrophages in the pancreas of a subject in need thereof. Generally, the method comprises administration of a therapeutically effective amount of a compound that disrupts the CSF-1 pathway, so as to substantially reduce CSF-1R signaling, selectively deplete islet macrophages in the pancreas, slow the progress of autoimmune diabetes, or delay the onset of autoimmune diabetes. Regarding the CSF-1 pathway, macrophage colony-stimulating factor, or CSF-1, acts on its target cells by binding to CSF-1R (c-fms), a member of the type III protein tyrosine kinase receptor family. A second ligand for CSF-1R is IL-34. Thus, two ligands (CSF-1 and IL-34) act on CSF-1R. Accordingly, the CSF-1 pathway comprises at least CSF-1, IL-34, and CSF-1R. Suitable compositions comprising a compound that disrupts the CSF-1 pathway are disclosed herein, for instance those described in Section I.

In another aspect, the disclosure encompasses a method of selectively depleting islet macrophages in the pancreas relative to tissue macrophages. The method comprises administering to a subject a composition comprising a compound that disrupts the CSF-1 pathway. A compound that disrupts the CSF-1 pathway may be a compound that binds to CSF-1, IL-34, and/or CSF-1R. For example, a compound that disrupts the CSF-1 pathway may be an anti-CSF-1 antibody, anti-IL-34 antibody, anti-CSF-1R antibody, or a small molecule tyrosine kinase inhibitor of CSF-1R. In certain embodiments, the compound binds to CSF-1R. In other embodiments, the compound is an anti-CSF-1R antibody.

By “selectively depleting” is meant that the reduction in islet macrophages is greater than the reduction in tissue macrophages following administration of a composition comprising a compound that targets the CSF-1 pathway. For example, a reduction may be measured using p-value. The reduction in islet macrophages is greater than the reduction in tissue macrophages when the p-value is less than 0.1, less than 0.05, less than 0.01, less than 0.005, or less than 0.001. Alternatively, a reduction may be measured using fold-change. The reduction in islet macrophages may be at least about 1.2-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 11-fold, at least about 12-fold, at least about 13-fold, at least about 14-fold, at least about 15-fold greater, or at least about 20-fold greater than the reduction in tissue macrophages.

In still another aspect, the disclosure provides a method of delaying the onset of autoimmune diabetes. The method comprises depleting islet macrophages in a subject. In certain embodiments, the method comprises depleting islet macrophages in a subject by administering to the subject a composition comprising a compound that disrupts the CSF-1 pathway. A compound that disrupts the CSF-1 pathway may be a compound that binds to CSF-1, IL-34, and/or CSF-1R. For example, a compound that disrupts the CSF-1 pathway may be an anti-CSF-1 antibody, anti-IL-34 antibody, anti-CSF-1R antibody, or a small molecule tyrosine kinase inhibitor of CSF-1R. In certain embodiments, the compound binds to CSF-1R. In other embodiments, the compound is an anti-CSF-1R antibody. The autoimmune diabetes may be latent autoimmune diabetes of adults (also referred to as LADA, late-onset autoimmune diabetes of adulthood, slow onset type 1 diabetes, or diabetes type 1.5) or type 1 diabetes.

In other aspects, the present disclosure provides a method of treating an inflammatory condition. The method comprises administering to a subject a composition comprising a compound that disrupts the CSF-1 pathway. In an embodiment, the compound binds to CSF1R. In another embodiment, the compound is an anti-CSF1R antibody.

A method of the disclosure may further comprise administering an anti-inflammatory agent.

### (a) Islet Macrophages

Islet macrophages reside in the pancreas and may also be referred to as islet resident macrophages (IRMs). The islet is a mini-organ that is constantly responding to several stimuli that vary and oscillate, such as blood glucose, the most prevalent bioactive molecule. The pancreas contains distinct resident macrophages in the exocrine and endocrine regions. In each anatomical site, the macrophages differ in their embryonal origin and in their activation status. In an analysis performed in C57BL/6 mice, the interacinar macrophages were found to express genes and cell surface markers that categorize them as M2-like (anti-inflammatory) and tissue supportive. In contrast, the macrophages in the islets of Langerhans expressed M1-like transcripts typically associated with inflammatory macrophages. For example, islet macrophages have been shown to express an activation signature with high expression of Tnf, II1b, and MHC-II at both the transcript and protein levels. Islet macrophages can be identified using methods disclosed herein, for example those used in the Examples below or as CD45⁺F4/80⁺MHC-II⁺ cells.

By “depleting islet macrophages” is meant that the amount of islet macrophages following administration of a composition comprising a compound that targets the CSF-1 pathway is reduced by at least about 1.2-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 11-fold, at least about 12-fold, at least about 13-fold, at least about 14-fold, at least about 15-fold, or at least about 20-fold less than the amount of islet macrophages prior to administration of a composition comprising a compound that targets the CSF-1 pathway.

### (b) Inflammatory Conditions

Non-limiting examples of inflammatory conditions include proliferative vascular disease, acute respiratory distress syndrome, cytokine-mediated toxicity, interleukin-2 toxicity, appendicitis, peptic, gastric and duodenal ulcers, peritonitis, pancreatitis, ulcerative, pseudomembranous, acute and ischemic colitis, diverticulitis, epiglottitis, achalasia, cholangitis, cholecystitis, hepatitis, inflammatory bowel disease, Crohn's disease, enteritis, Whipple's disease, allergy, anaphylactic shock, immune complex disease, organ ischemia, reperfusion injury, organ necrosis, hay fever, cachexia, hyperpyrexia, eosinophilic granuloma, granulomatosis, sarcoidosis, pulmonary sarcoidosis, septic abortion, epididymitis, vaginitis, prostatitis, urethritis, bronchitis, emphysema, rhinitis, cystic fibrosis, pneumonitis, alvealitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, cerebral malaria, filariasis, amebiasis, hydatid cysts, burns, dermatitis, contact dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, celiac disease, congestive heart failure, meningitis, encephalitis, cerebral infarction, cerebral embolism, Guillain-Barre syndrome, neuritis, neuralgia, spinal cord injury, paralysis, uveitis, arthritides, arthralgias, osteomyelitis, fasciitis, Paget's disease, periodontal disease, myasthenia gravis, thryoiditis, lupus, including systemic lupus erythematosus, lupus nephritis, and cutaneous lupus, Goodpasture's syndrome, Behcets's syndrome, allograft rejection, graft-versus-host disease, Berger's disease, Berger's disease, Retier's syndrome, Hodgkins disease, arthritis, psoriatic arthritis, Reiter's syndrome, gout, traumatic arthritis, rubella arthritis and acute synovitis, rheumatoid arthritis, rheumatoid spondylitis, ankylosing spondylitis, osteoarthritis, gouty arthritis and other arthritic conditions, sepsis, septic shock, endotoxic shock, gram negative sepsis, toxic shock syndrome, Alzheimer's disease, stroke, neurotrauma, asthma, chronic pulmonary inflammatory disease, silicosis, bone resorption disease, osteoporosis, restenosis, thrombosis, glomerularonephritis, ulcerative colitis, multiple sclerosis, muscle degeneration, eczema, psoriasis, and conjunctivitis. In addition, the disclosure provides a method of treating inflammation associated with any of the aforementioned inflammatory conditions.

### (c) Subject

As used herein, “subject” or “patient” is used interchangeably. Methods described herein are generally performed on a subject in need thereof. Suitable subjects include, but are not limited to, a human, a livestock animal, a companion animal, a lab animal, and a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas, and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In specific embodiments, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In certain embodiments, the subject is a human, a livestock animal, or a companion animal. In a specific embodiment, the subject is a human.

### III. Kits

Also provided are kits. Such kits can include a composition described herein and, in certain embodiments, instructions for administration. Such kits can facilitate performance of the methods described herein. When supplied as a kit, the different components of the composition can be packaged in separate containers and admixed immediately before use. Components include, but are not limited to compositions and pharmaceutical formulations comprising a compound that disrupts the CSF-1 pathway. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

### Definitions

When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements.

The term “treat,” “treating,” or “treatment” as used herein refers to administering a pharmaceutical composition of the disclosure for prophylactic and/or therapeutic purposes. The term “prophylactic treatment” refers to treating a subject who does not yet have disease, but who is susceptible to, or otherwise at a risk of disease. The term “therapeutic treatment” refers to administering treatment to a subject already suffering from disease. The term “treat,” “treating,” or “treatment” as used herein also refers to administering a pharmaceutical composition of the disclosure in order to: (i) reduce or eliminate either disease or one or more symptoms of the disease, or (ii) retard the progression of disease or of one or more symptoms of disease, or (iii) reduce the severity of disease or of one or more symptoms of disease, or (iv) suppress the clinical manifestation of disease, or (v) suppress the manifestation of adverse symptoms of the disease. The terms “inhibition” or “inhibit” refer to a decrease or cessation of any phenotypic characteristic or to the decrease or cessation in the incidence, degree, or likelihood of that characteristic. The term “control” or “controlling” as used herein generally refers to preventing, reducing, or eradicating disease or inhibiting the rate and extent of such a disease, or reducing the inflammation, wherein such prevention or reduction in the disease is statistically significant with respect to untreated disease.

The term “effective amount” as used herein refers to an amount, which has a therapeutic effect or is the amount required to produce a therapeutic effect in a subject. For example, a therapeutically or pharmaceutically effective amount of a composition is the amount of the compound required to produce a desired therapeutic effect as may be judged by clinical trial results and/or model animal studies. The effective or pharmaceutically effective amount depends on several factors, including but not limited to, the route of administration, the disease involved, characteristics of the subject (for example height, weight, sex, age and medical history), severity of disease, location of disease, and/or the particular type of compound used. For prophylactic treatments, a therapeutically or prophylactically effective amount is that amount which would be effective to prevent disease.

As used herein, an “anti-inflammatory agent” is an agent that blocks the production of certain chemicals in the body that cause inflammation.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. Depletion of Islet Macrophages with CSF-1R Antibody

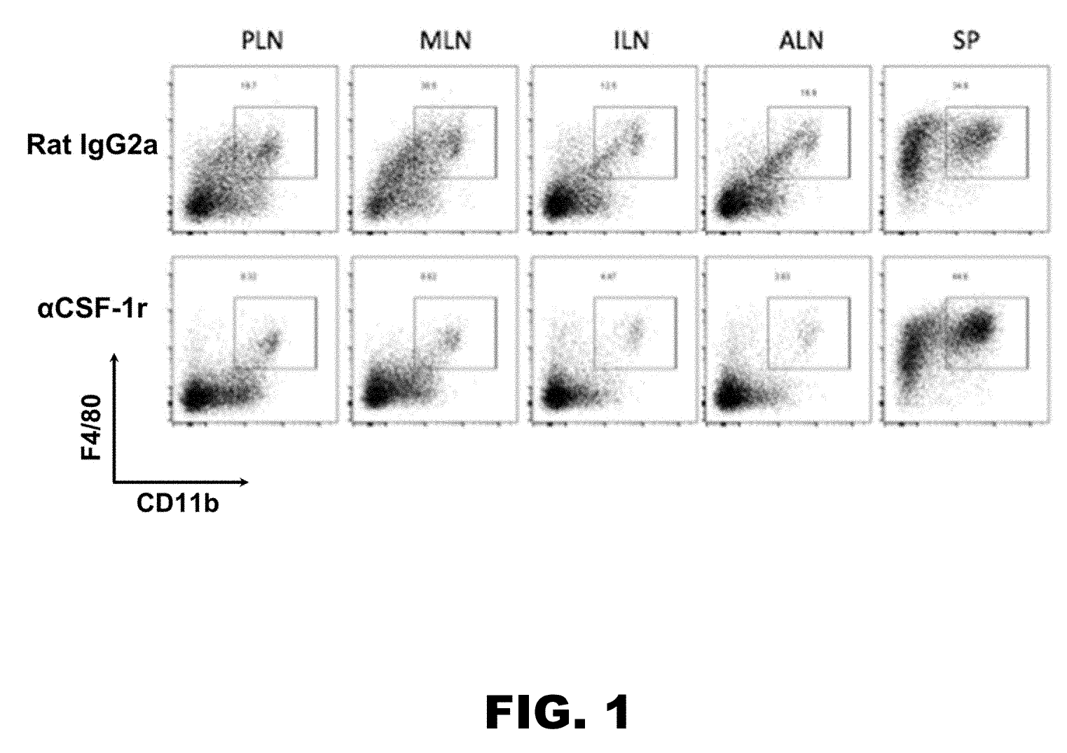

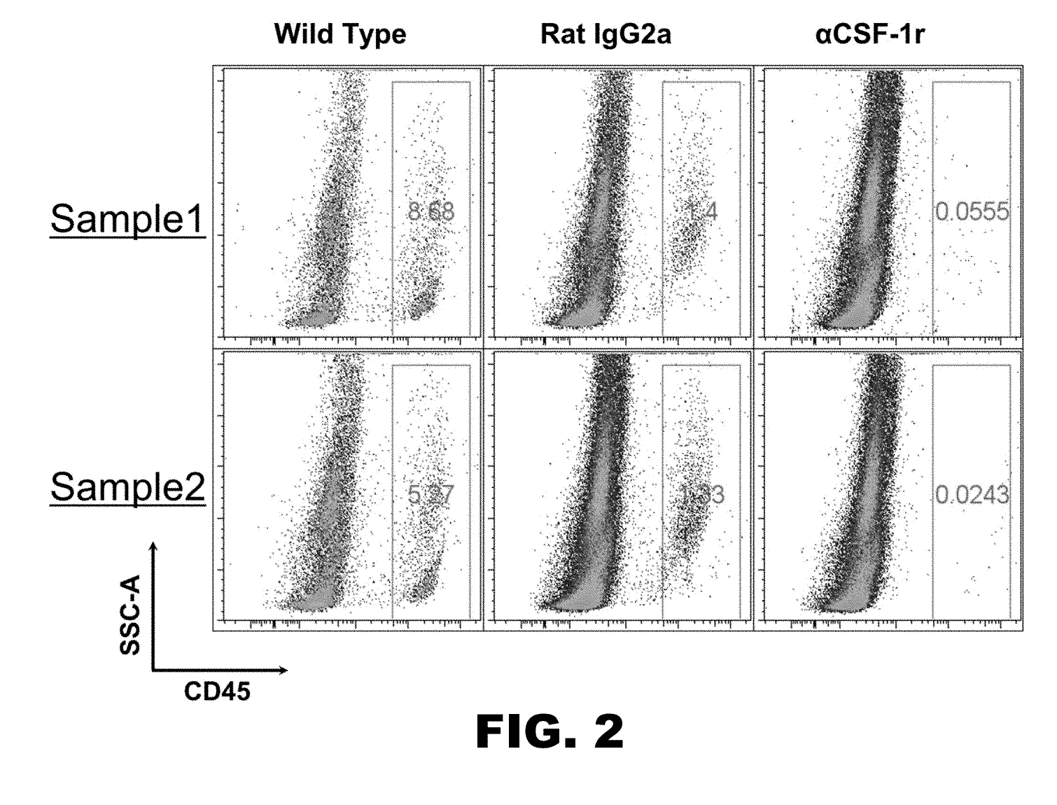

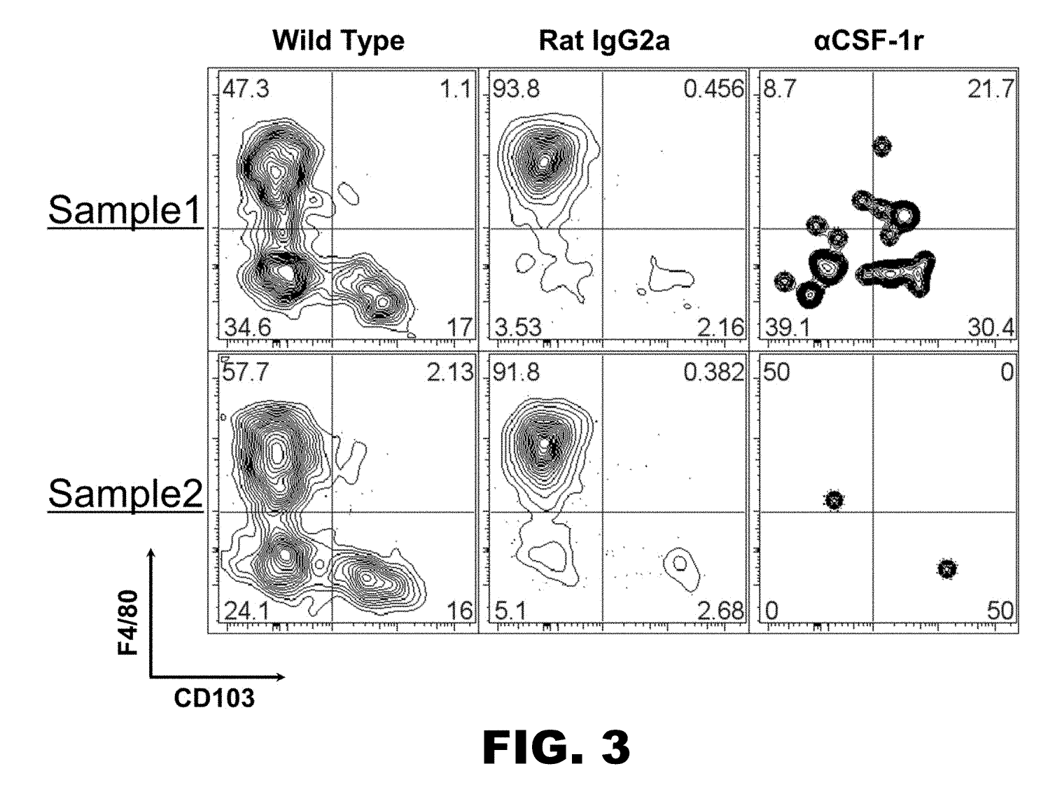

To determine if antibody mediated blockade of the CSF-1R can deplete the intra-islet macrophage, three-week old non-obese diabetic NOD.RAG mice were treated with 0.5 mg AFS98 per week. Islets, lymph nodes and peritoneum lavage were harvested for analysis of the macrophage populations at 6 weeks. It was determined via flow cytometry that αCSF-1R treatment for 3 weeks caused a 3-fold reduction in lymph node resident macrophages (FIG. 1). Further, αCSF-1R treatment for 3 weeks depleted intra-islet leukocytes (FIG. 2). More specifically, it was shown that αCSF-1R treatment for 3 weeks depleted intra-islet macrophages (FIG. 3).

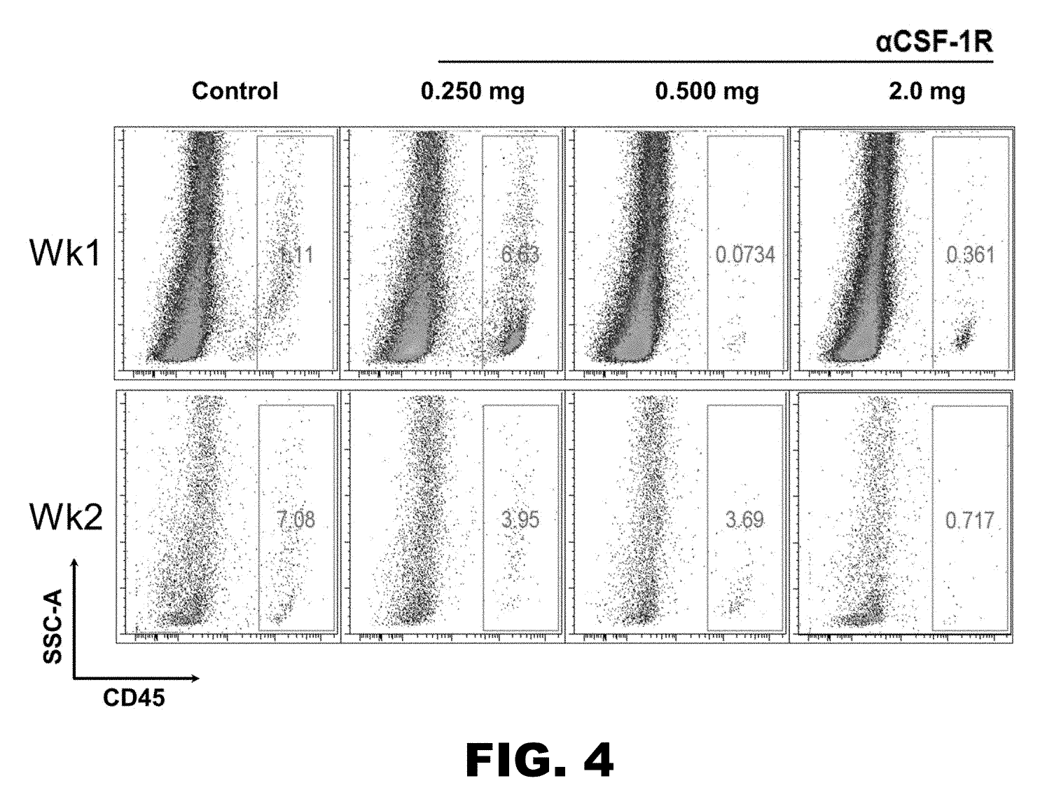

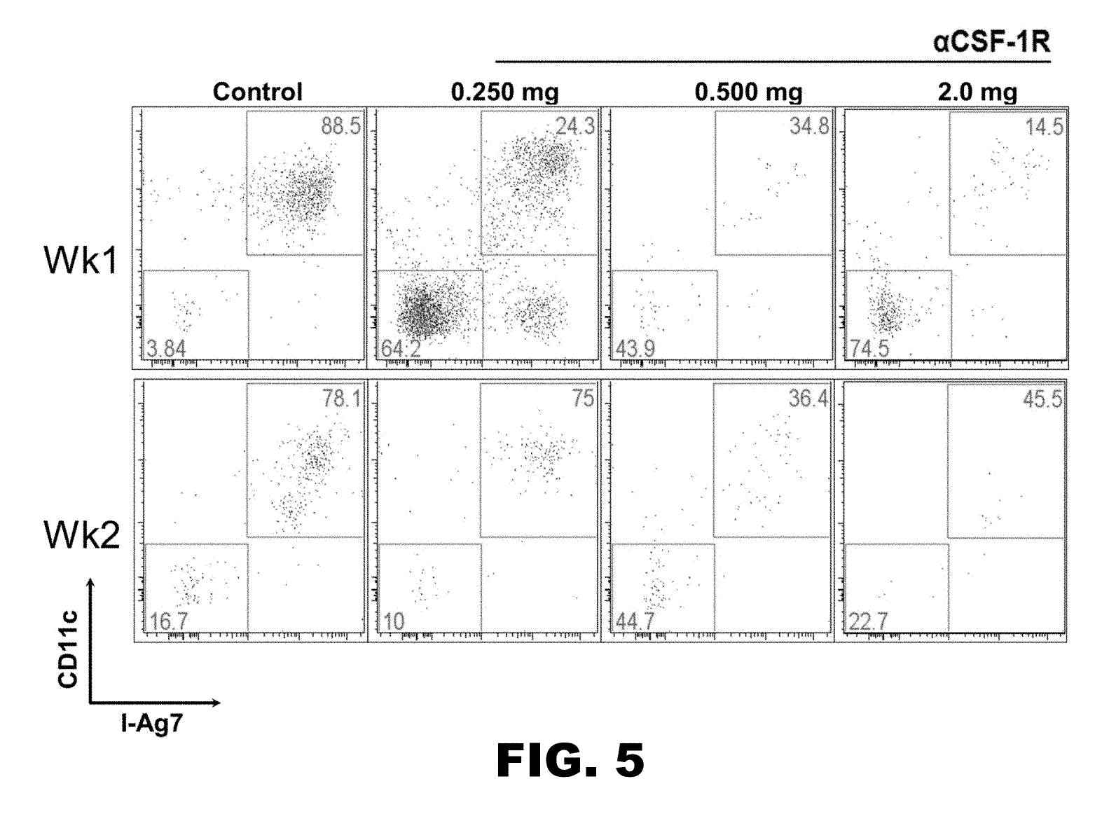

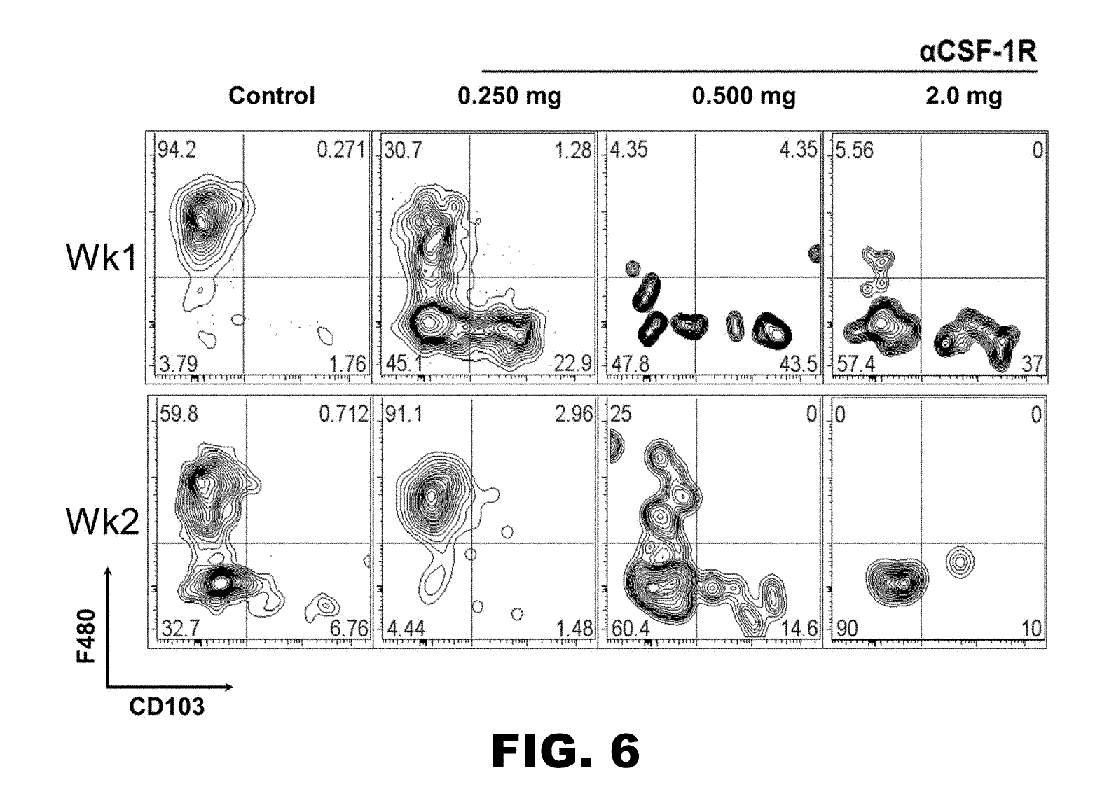

The dose of αCSF-1R was titrated to determine the optimal dose for depletion during inflammation. Five- to six-week old wild-type NOD mice were treated with a single of dose of AFS98 antibody at 0.25 mg, 0.5 mg, or 2.0 mg. Islets, lymph nodes and peritoneum lavage were harvested for analysis of the islet infiltration populations at 7 and 14 days following treatment. It was determined that the intra-islet macrophages remained depleted at 2 weeks following treatment with the αCSF-1R antibody at 0.25 mg, 0.5 mg and 2.0 mg (FIG. 4, FIG. 5, and FIG. 6).

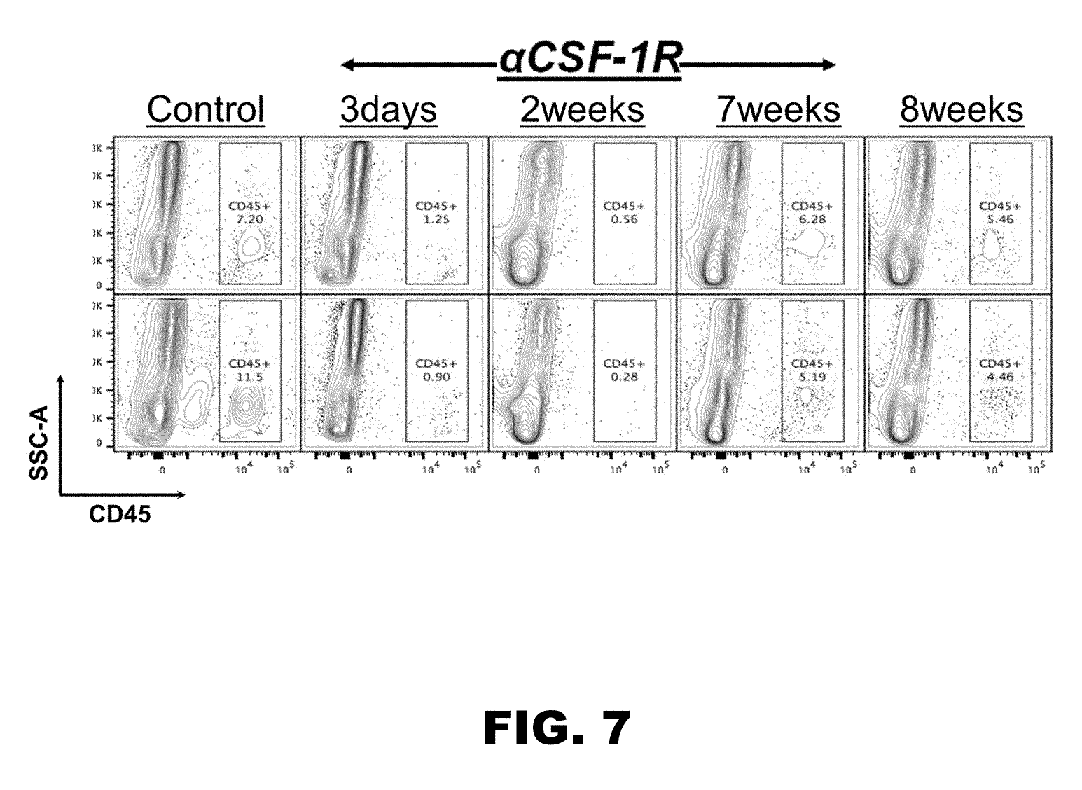

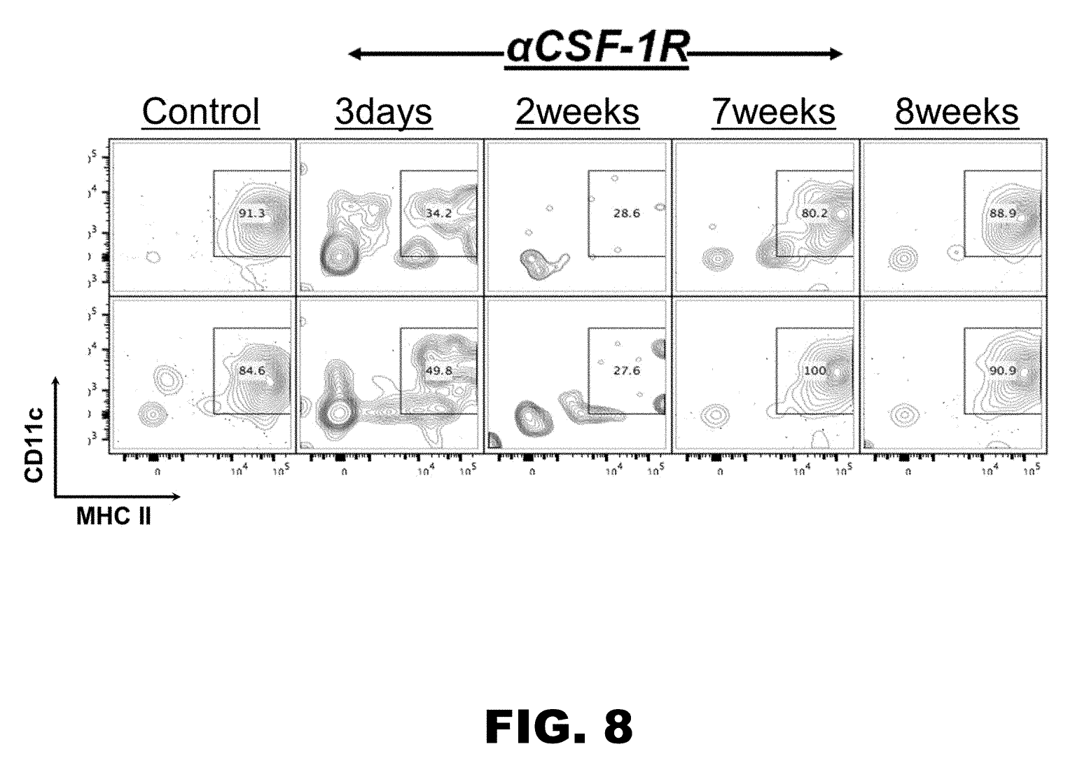

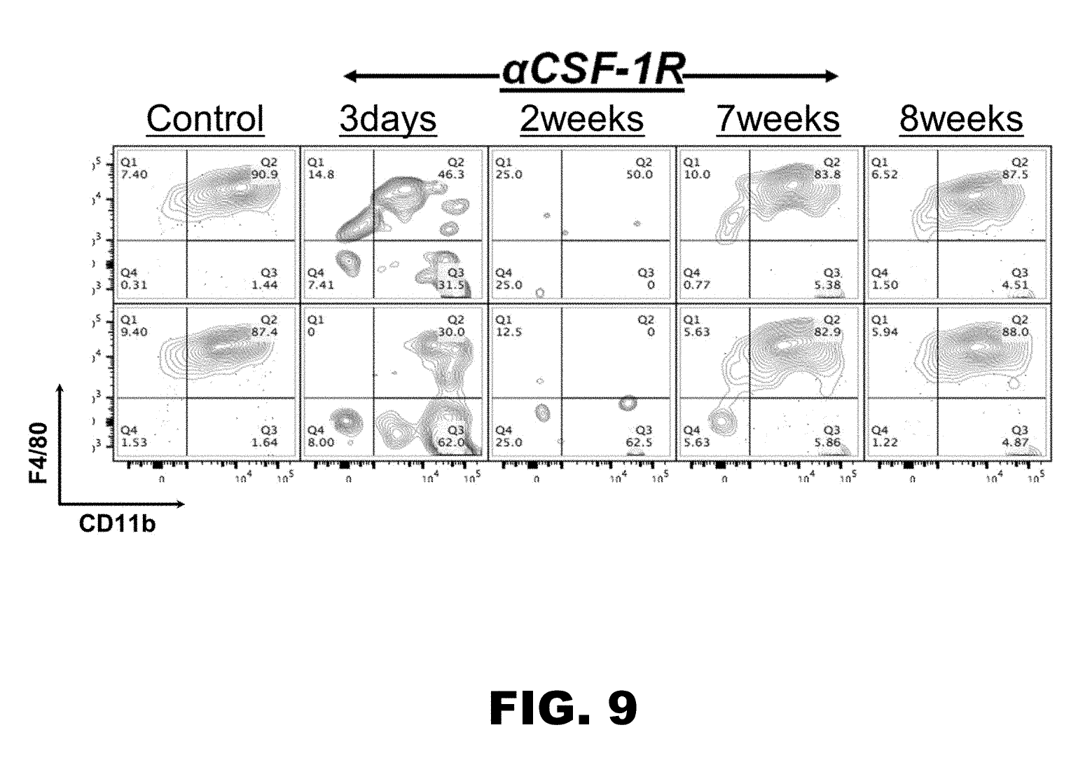

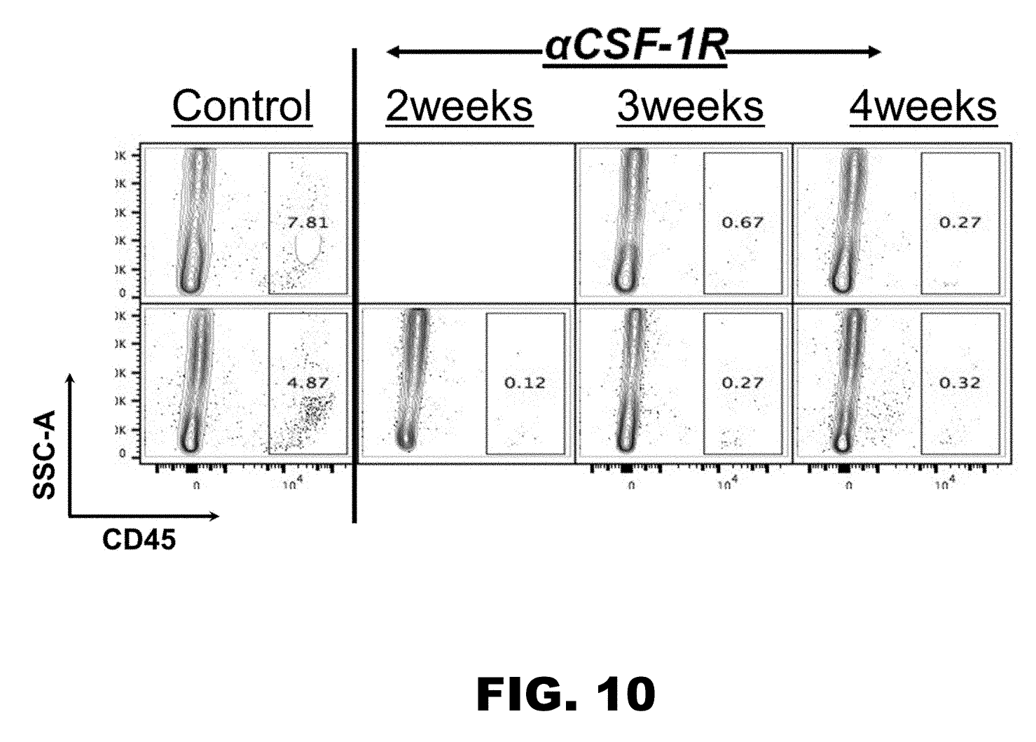

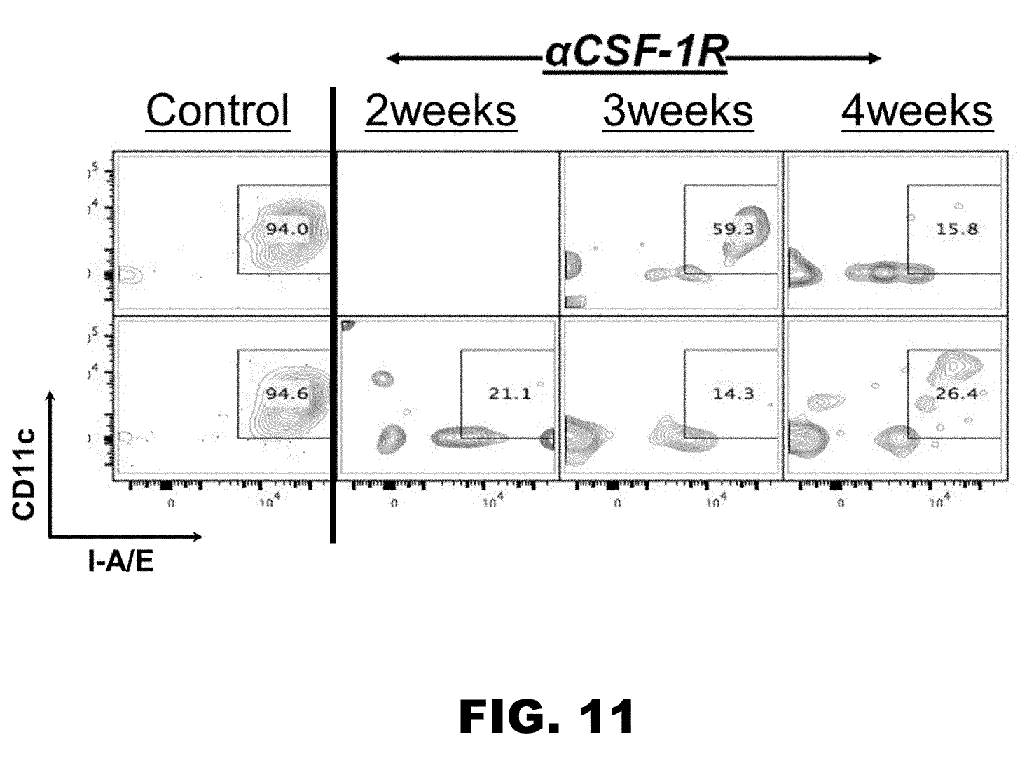

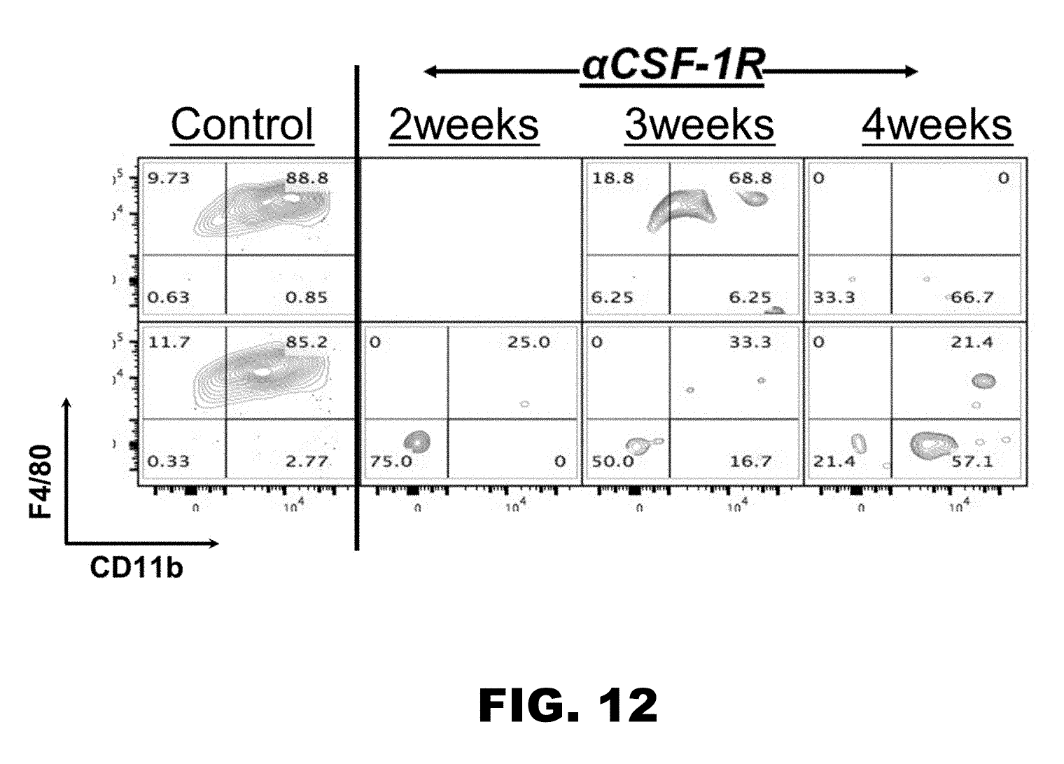

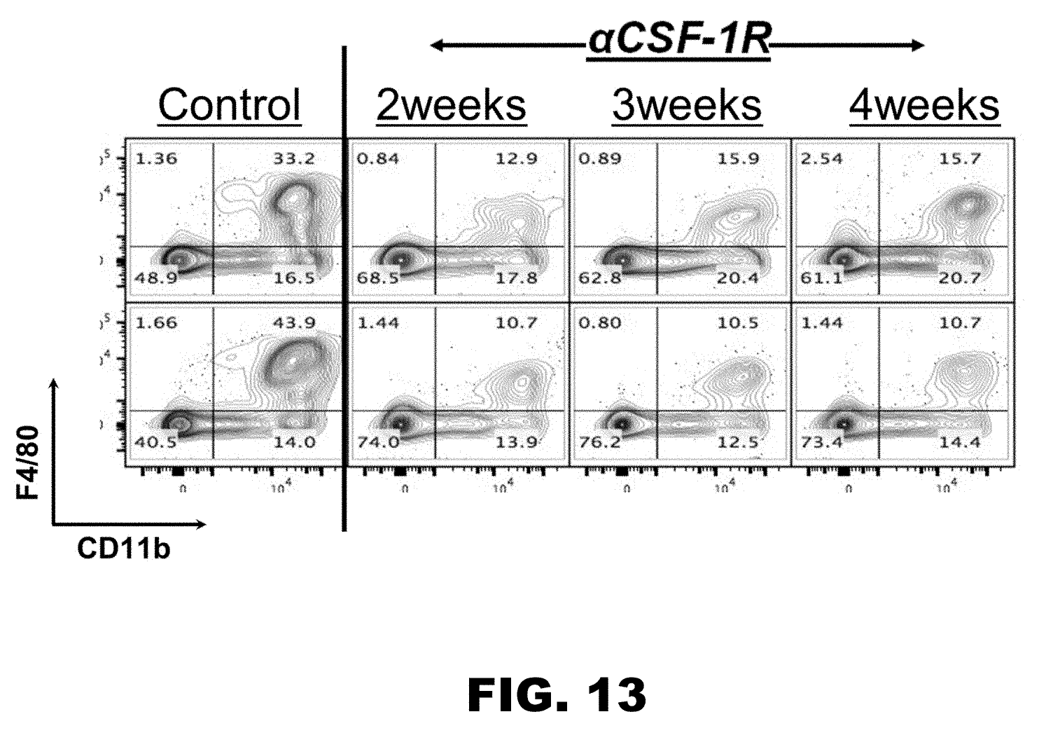

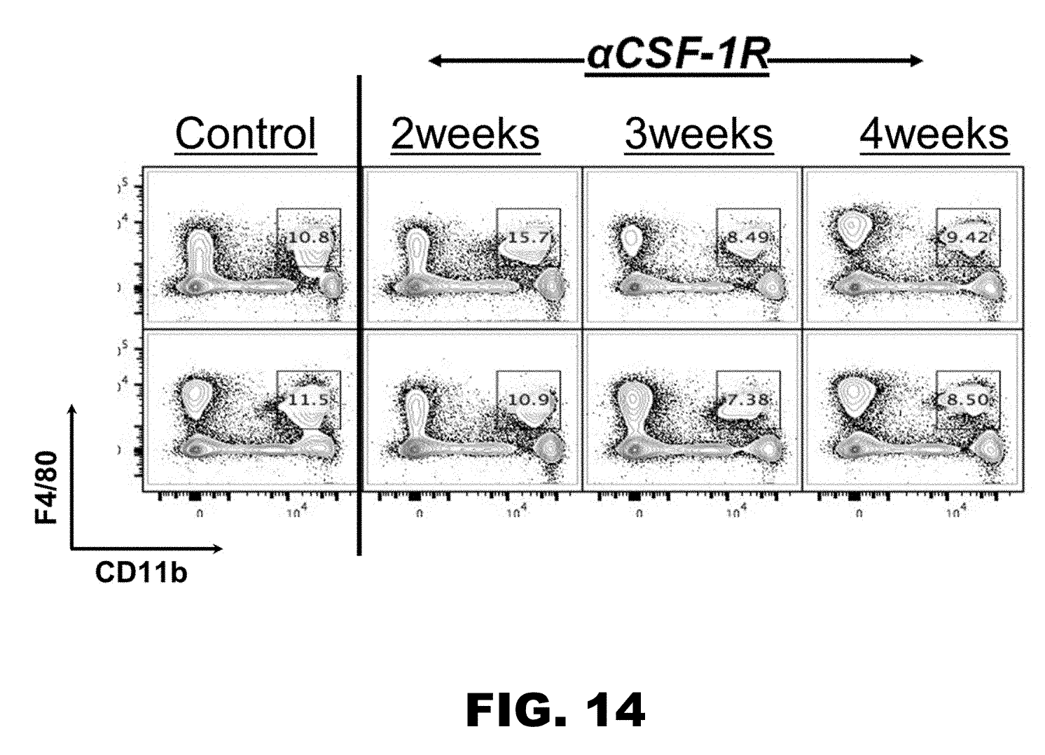

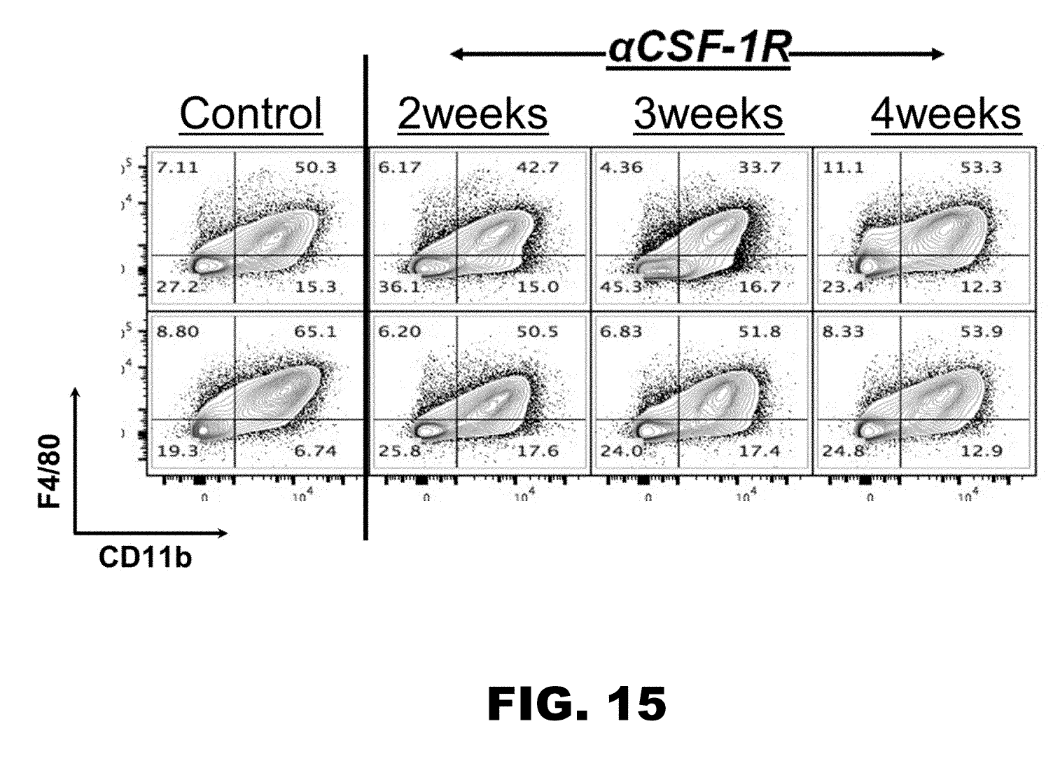

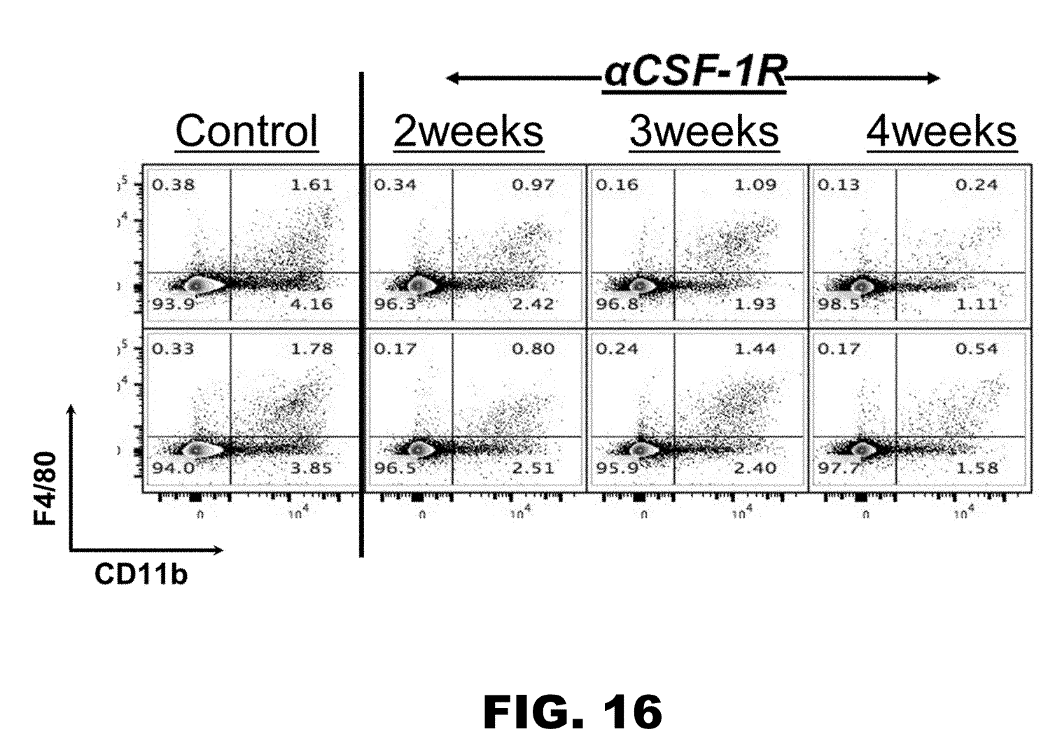

Next, how the islet macrophages affect islet/β-cell homeostasis was evaluated as well as how long the depletion lasts. Parameters used to determine this included histology to evaluate islet size and mass, an RIA assay to evaluate insulin protein, a glucose tolerance assay, electron microscopy to determine morphology and ER stress and a microarray to analyze transcript levels. C57BL/6 mice were treated with a single dose of 2.0 mg AFS98 antibody. Islets, lymph nodes, and stroma were harvested for analysis of macrophages by flow cytometry at 2 weeks, 4 weeks, 6 weeks, 8 weeks, and 10 weeks following treatment. Upon examining the islet macrophage population at 3 days, 2 weeks, 7 weeks, and 8 weeks, it was observed that following a single dose of αCSF-1R antibody at 2.0 mg peak depletion was at 2 weeks with depletion evident throughout 8 weeks (FIG. 7, FIG. 8, and FIG. 9). A second experiment, confirmed the longevity of islet macrophage depletion, with significant decreases relative to control observed at 2 weeks, 3 weeks, and 4 weeks (FIG. 10, FIG. 11, and FIG. 12). The amount of stromal, lung, splenic and lymph node were also determined via flow cytometry. The results showed that macrophage depletion is also observed in pancreatic stroma (FIG. 13), but much less in lung tissue (FIG. 14), splenic tissue (FIG. 15), and the lymph nodes (FIG. 16). This is a good sign of selectivity of the αCSF-1R antibody. Specifically, the islet macrophages are very sensitive to depletion in contrast to those in other tissues.

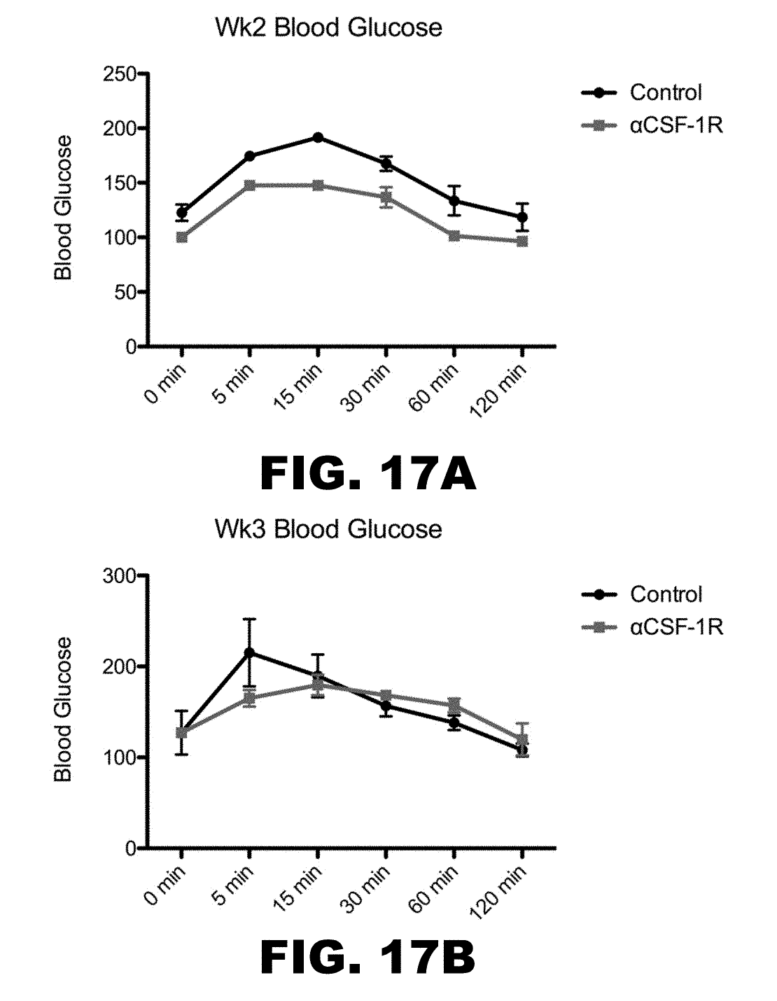

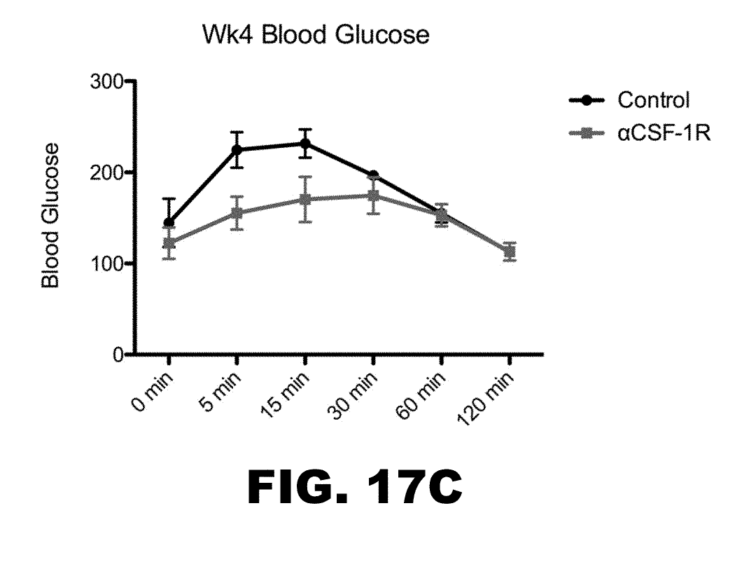

In addition to evaluating macrophage depletion, a glucose tolerance test was performed 1 day prior to each harvest at a 12 hour fast. Blood glucose levels were evaluated at 2 weeks, 3 weeks, and 4 weeks following administration of the αCSF-1R antibody. The mice treated with αCSF-1R antibody did not show a detrimental effect on the function of pancreatic islets. Their response to glucose was normal (FIG. 17) as well as their content of pancreatic insulin (control antibody treated, 69 ng/gm of pancreatic tissue; CSFR1 treated, 0.049 ng/gm of pancreatic tissue). Differences were not statistically different.

### Example 2. αCSF-1R Antibody Blocks the Progression of Diabetes

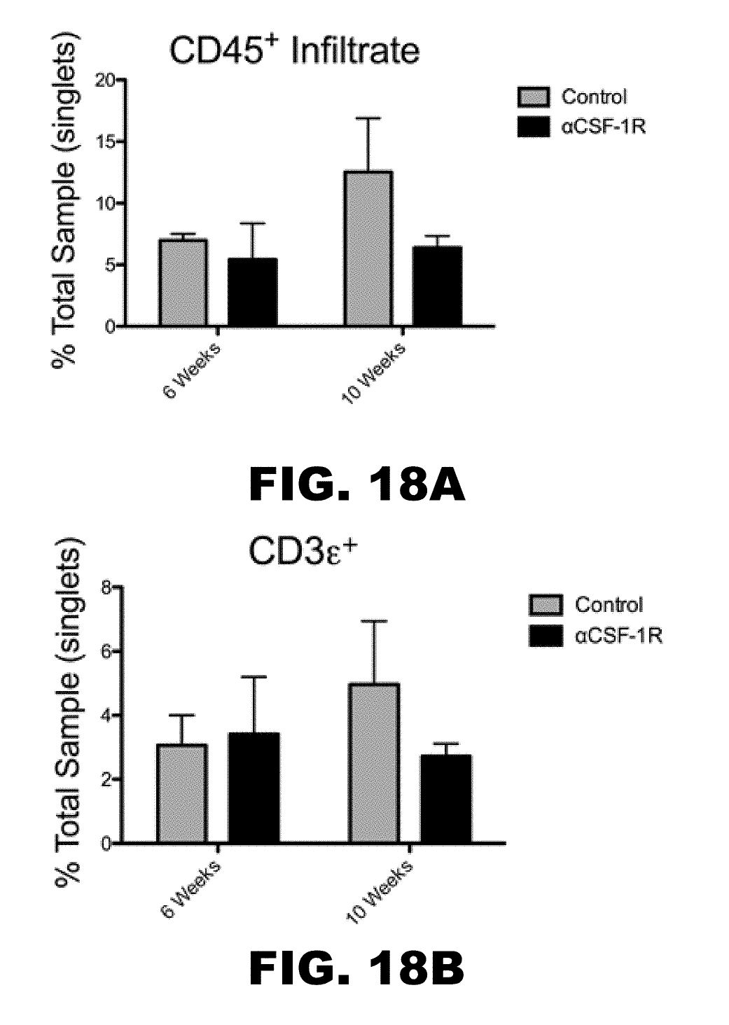

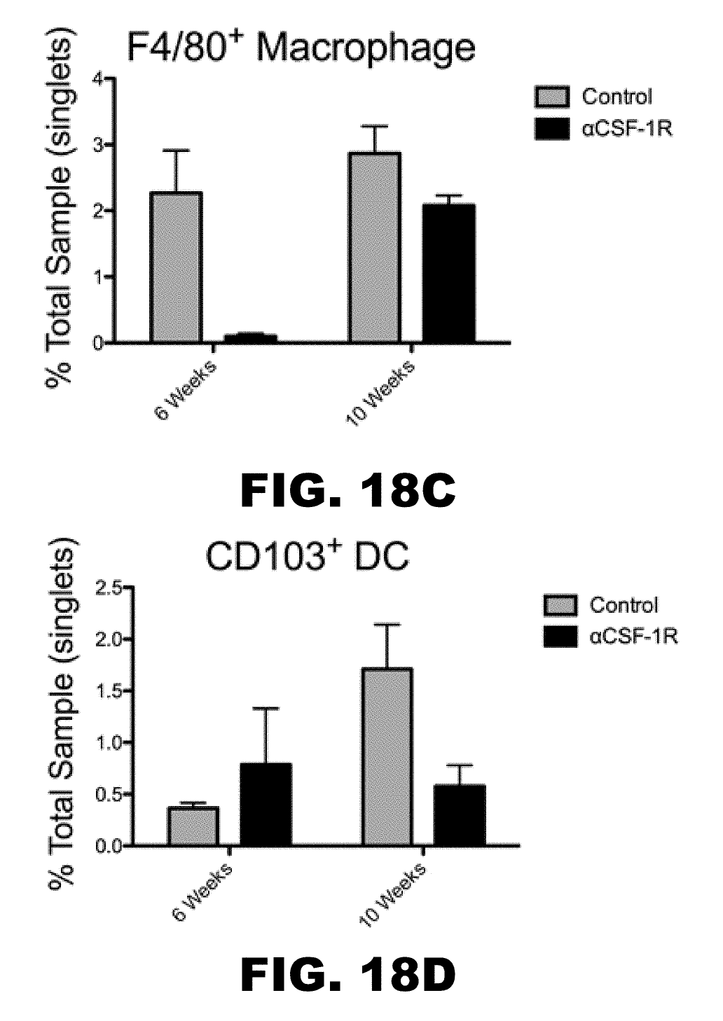

To determine if treatment with αCSF-1R antibody can be used to block the progression of diabetes, three-week old wild-type NOD mice, a diabetic strain, were treated with 0.5 mg of AF298 per week for three weeks. Islets, lymph nodes and peritoneum lavage were harvested for analysis of islet macrophage populations by flow cytometry at 6 weeks and 10 weeks following initiation of treatment. Macrophages were significantly depleted at 6 weeks and 10 weeks post-initiation of treatment (FIG. 18).

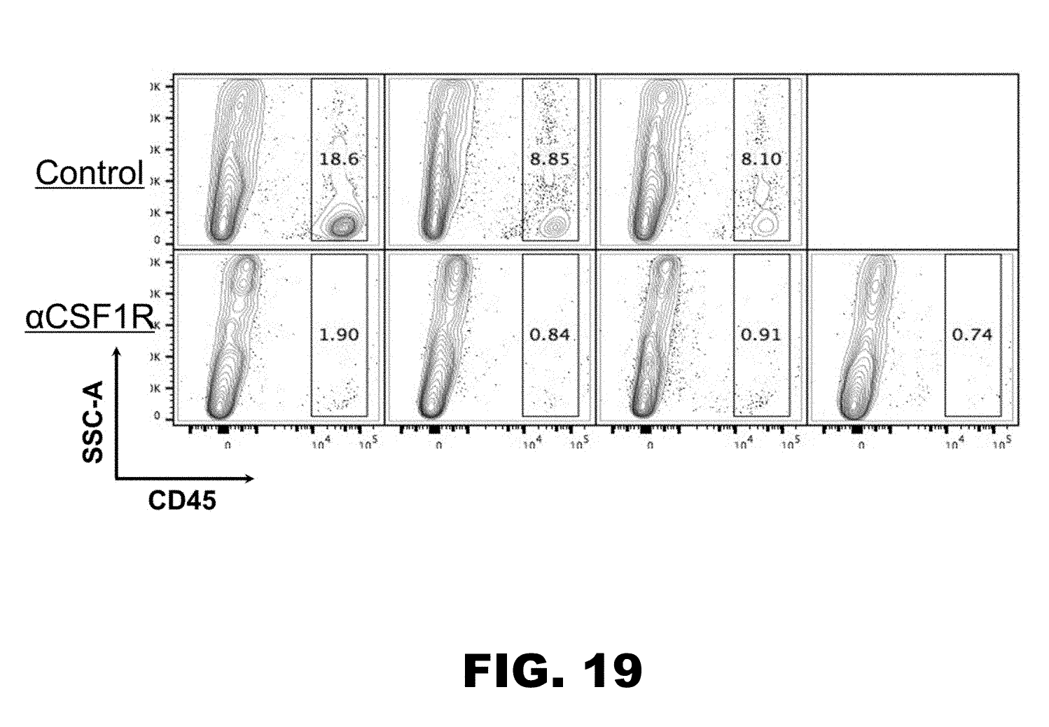

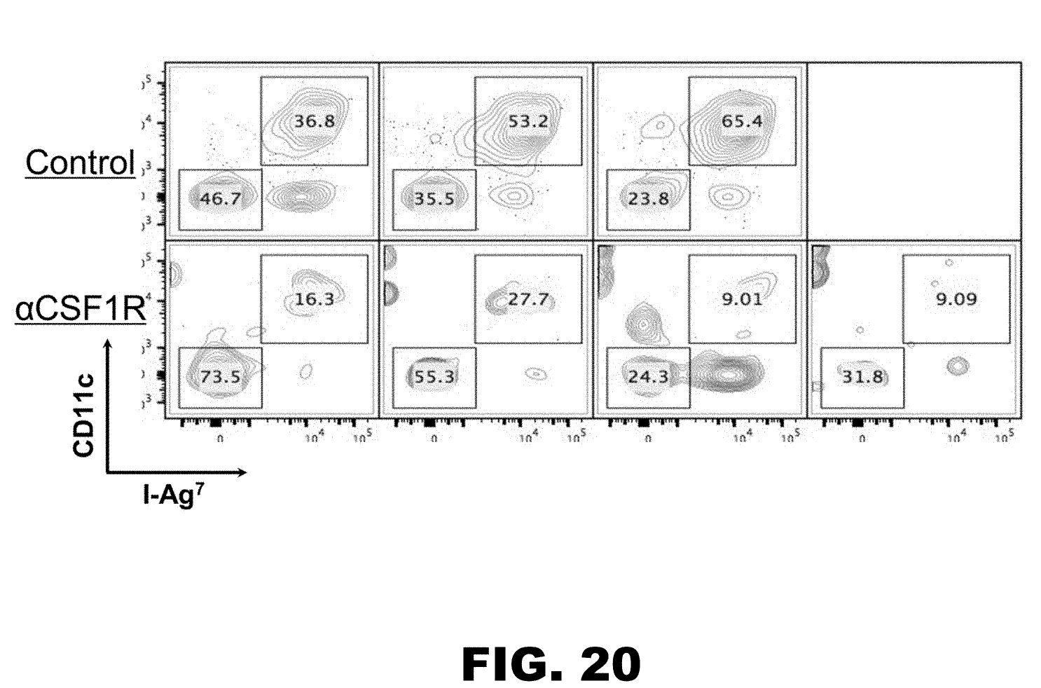

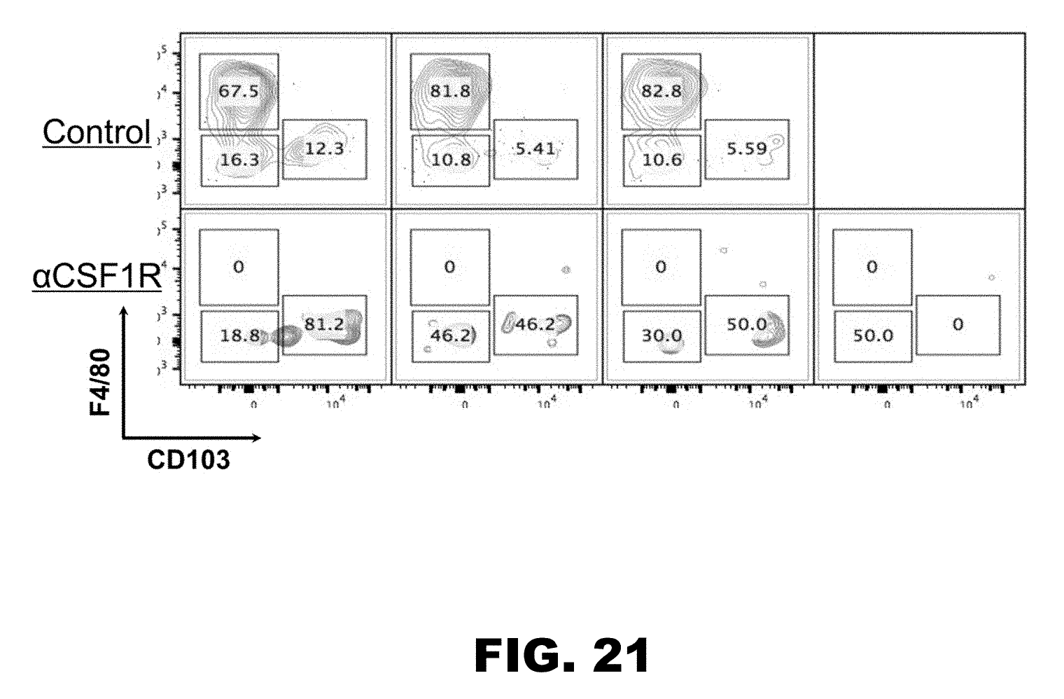

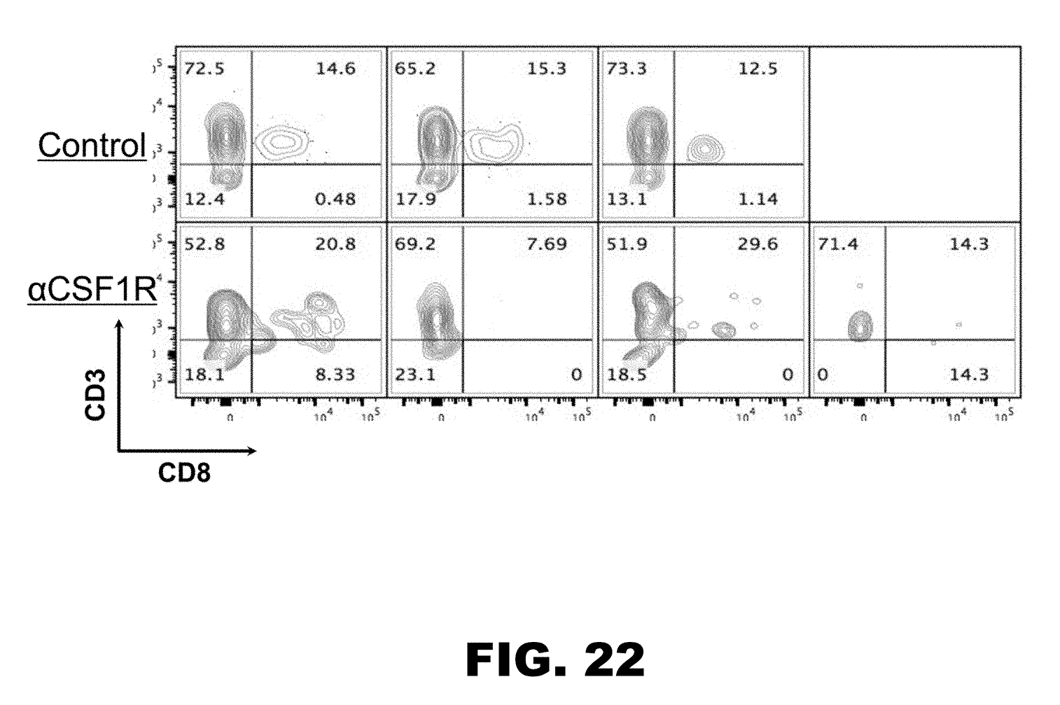

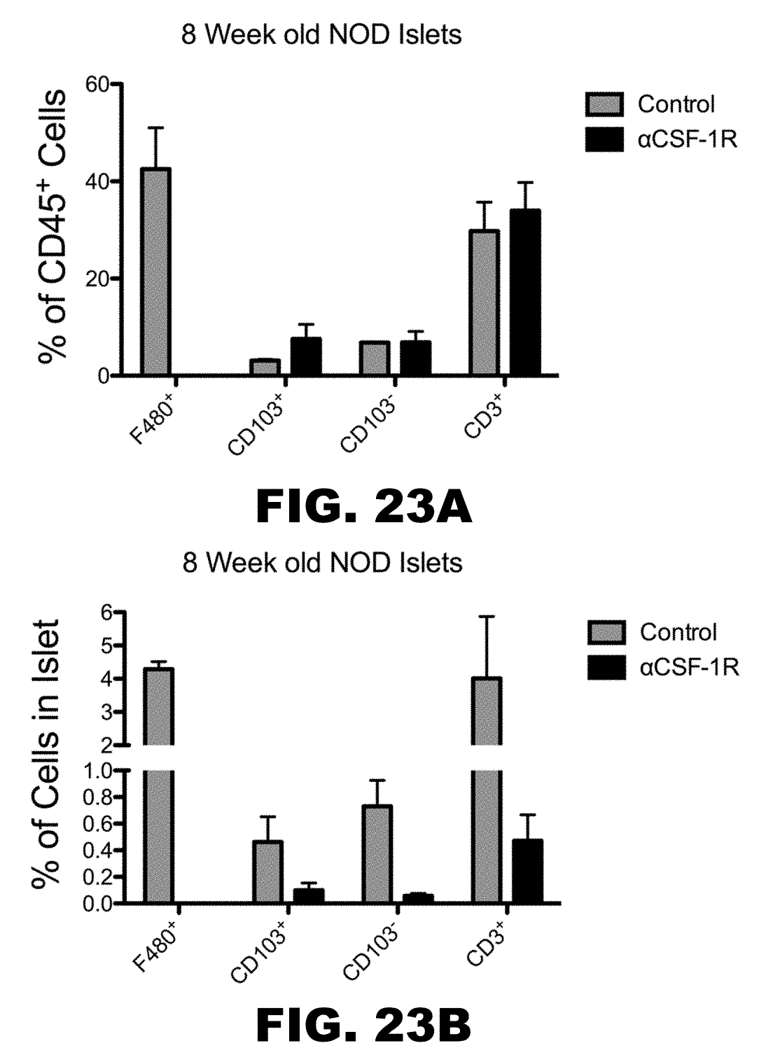

In a separate experiment, two-week old wild-type NOD mice were treated with 0.5 mg of AF298 and then an additional 2.0 mg of AF298 at 3 weeks, 6 weeks, 9 weeks, and 12 weeks of age. Tissues were sampled for immune profiling of islet macrophages by flow cytometry. Flow cytometry plots show a reduction in islet macrophages at 2 weeks, 3 weeks, and 6 weeks post-initiation of treatment (FIG. 19, FIG. 20, FIG. 21, and FIG. 22). Results show that not only does inhibition of CSF-1R signaling leads to a depletion of the intra-islet macrophage, anti-CSF-1R treatment may also limit the accumulation of immune cells in the islets (FIG. 23). In diabetes, the entrance of lymphocytes is an index of on-going disease. In line with this, 8 of 10 control treated mic were diabetic at 40 weeks of age whereas in mice receiving anti-CSF-1R antibody only 2 of 11 mice were diabetic. In a separate experiment, at 3 weeks of observation 3 out of 10 mice were diabetic in control treated mice, wherein none of the anti-CSF-1R antibody-treated mice were diabetic. Accordingly, this suggests that the ability to inhibit CSF-1R signaling leads to a reduction in the accumulation of immune cells in the islets which leads to a reduction in the incidence of diabetes.

### Example 3. Macrophages of Pancreatic Islets have a Seminal Role in the Initiation of Autoimmune Diabetes of NOD Mice

### Introduction

Tissue-resident macrophages constitute a heterogeneous and multifunctional cell type that populates virtually every tissue (1, 2). In addition to their primary role as sentinels for invading pathogens and as initiators of inflammation, macrophages have important homeostatic roles in many tissue-specific processes such as development, metabolism, and tissue remodeling, providing trophic factors within the tissue microenvironment. Their homeostatic role is evidenced by the deleterious phenotype that arises when macrophage development is genetically constrained. The osteopetrotic mouse (op/op) harbors a severe paucity of macrophages due to a null mutation in the gene encoding the macrophage differentiation and survival factor, colony stimulating factor-1 (CSF-1) (3). These mice present with a number of developmental defects. Notably, the islets of Langerhans, which normally contain a population of CSF-1-dependent macrophages, are atrophic in the op/op mouse, suggesting an integral role for macrophages during the development of the endocrine pancreas (4, 5).

The pancreatic islets of all species contain a small number of myeloid cells represented by macrophages. These macrophages were first identified by immunohistochemical analysis using macrophage-specific markers. Although initially thought to be “passenger leukocytes,” subsequent studies using lineage tracing approaches identified them as resident cells (5). These self-replicate and are replaced minimally, if at all, by blood monocytes. Islet macrophages are highly activated with a complex gene transcriptome reflecting their interactions with beta cells and with blood components (6). They express high levels of class II histocompatibility molecules (MHC-II) and a number of cytokines and chemokines including TNF-α and IL-113 (5, 6). The islet macrophages are always next to blood vessels, in close contact with beta cells, capture insulin-containing granules, and present insulin peptides to autoreactive CD4 T cells (7). Moreover, macrophages extend filopodia into the vessel lumen and can respond to blood stimuli (6, 8).

Our laboratory focuses on the early events that initiate autoimmune diabetes as exemplified in the NOD mouse strain. Understanding the initiation of autoimmune diabetes is of considerable clinical interest. Along those lines, identifying the key cellular and molecular events that transition the immune-privileged islet to an immunologically reactive environment is paramount as it pertains to diabetes initiation. By 3 weeks of age, islets show significant cellular and molecular changes compared with nondiabetic strains: an early entrance of CD4⁺ T cells reactive to insulin in contact with the macrophages, a heightened gene signature of activation in the macrophages, and the appearance of the XCR1⁺ subset of dendritic cells (DCs) (9, 10). The deficit of XCR1⁺ DCs in the NOD Batf3^{-/-} mice translates into a profound reduction in the autoimmune process (10).

Here, the role of the resident macrophages in diabetogenesis was examined. The islet macrophage expresses both inhibitory and stimulatory ligands and receptors, as well as chemokines and chemokine receptors (6). They are the sole hematopoietic cell within the islets before 3 weeks of age and are able to present unconventional insulin epitopes to autoreactive T cells (6). Altogether, this posits the intraislet macrophage as a key regulator of lymphocyte entry into the islet. They have been eliminated using a monoclonal antibody to the CSF-1 receptor (CSF-1R), AFS98 (11). This monoclonal antibody, and the M279 antibody against CSF-1R, when administered in vivo resulted in the loss of macrophages from many tissues (12-14). The extent of depletion depended on the amount and frequency of administration (12). Although treatment with AFS98 resulted in the loss of resident macrophages, blood monocyte counts and inflammatory responses were not affected (13). Presented herein is evidence that the anti-CSF-1R antibody AFS98 depleted the islet resident macrophages in two strains of mice: the normal C57BL/6 (B6) and the diabetogenic NOD. The depletion of macrophages did not affect the metabolism or islet transcriptome of the mouse. However, diabetes progression was severely blunted.

### Methods and Materials

Mice.

Female C57BL/6 mice (B6), female NOD/ShiLtJ (NOD), NOD. 129S7(B6)-Rag1^{tm1Mom}/J (NOD.Rag1^{-/-}), NOD.Cg-Tg(TcraBDC2.5,TcrbBDC2.5)1Doi/DoiJ (BDC2.5), and NOD.Cg-Tg(TcraTcrbNY8.3)1Pesa/DvsJ (NY8.3) mice were purchased from the Jackson Laboratories (Bar Harbor, Me.) and bred in house. NY8.3 mice were backcrossed onto the congenic NOD.B6-Ptprc^{b}/6908MrkTacJ (NOD.CD45.2) background for T cell transfer experiments (18). All mouse experiments were performed in accordance with the Division of Comparative Medicine of Washington University School of Medicine [Association for Assessment and Accreditation of Laboratory Animal Care (AAALAC) accreditation no. A3381-01].

T Cell Transfer Assay;

ELISPOT; Anti-PD-1 Treatment. 8F10, BDC2.5, and NY8.3 TCR transgenic mouse spleens and lymph nodes were harvested and dispersed into single-cell suspensions. CD4- or CD8-positive cells were selected via magnetic microbead cell separation (Miltenyi Biotech, Bergisch Gladbach, Germany) according to the manufacturer's protocol. The cells were then stained with 1 μM carboxyfluorescein diacetate succinimidyl ester (Life Technologies, Carlsbad, Calif.) for 15 minutes at 37° C. and immediately quenched with 4° C. DMEM supplemented with 10% FCS. CFSE-labeled cells (3.0×10⁶) were then transferred into recipient mice via tail vein injections. Islets, draining pancreatic lymph nodes, and inguinal lymph nodes were harvested, dispersed into single-cell populations, and stained with fluorescent antibodies for analyses by flow cytometry. For experiments dealing with anti-PD-1, AFS98-treated or control NOD mice were injected with 200 μg of antibody three times over 6 days (days 0, 3, and 6; Anti-PD-1 clone RMP1-4; Leinco Technologies, Fenton, Mo.). Mice were then followed for diabetes incidence. For experiments examining the lymph node response by ELSIPOT, B6.g7 or NOD mice (7-10 weeks old) were treated with a single 2.0-mg dose of AFS98 or control rat IgG2a. Seven days later, the mice were immunized in the footpad with 10 nmol INS B:9-23 peptide, HEL protein (Sigma-Aldrich), or IGRP:206-214 peptide. After 7 days, the animals were killed and the popliteal lymph nodes were removed and made into single-cell suspensions. Lymph nodes were assayed for reactivity by eliciting a recall response on either IL-2 or IFN-γ-coated 96-well multiscreen plates (MilliporeSigma) for ELISPOT. One million popliteal lymph node cells were plated per well with 10 μM antigen, and reactive cells were counted using IMMUNOSPOT Software (Cellular Technology Limited (CTL), Cleveland, Ohio). Immune responses in mice challenged with INS B:9-23 peptide were recalled with either insulin or INS B:9-23. Immune responses in mice challenged with HEL protein were recalled with either HEL protein or HEL peptides 11-25 (CD4 epitope) or 20-35 (CD8 epitope). Finally, responses in mice challenged with IGRP:206-214 peptide were recalled with either IGRP:128-142 (CD4 epitope) or IGRP:206-214 (CD8 epitope).

Glucose Tolerance Assay and Insulin Quantitation.

For glucose tolerance experiments, female B6 mice were maintained on a normal diet and then fasted overnight for 12 hours. Fasting blood glucose levels were tested before a challenge with 1.5 g/kg glucose in PBS (Life Technologies) administered by i.p. injection. Blood glucose was measured at the intervals indicated in the experiments. For insulin quantitation, control or αCSF-1R-treated mice were maintained on 20% sucrose water for 7 days, returned to regular water for an additional 2 days, fasted overnight, and then their pancreata were removed and weighed. Pancreata was minced and homogenized. The supernatant was collected after pelleting the homogenate, and insulin was quantified using the Rat Insulin Radioimmune Assay (MilliporeSigma), as per the manufacturer's instruction.

Antigen Presentation Assay.

Islets were dispersed nonenzymatically, and 4.0×10⁴ islet cells per well were plated together in a 96-well plate with 5.0×10⁴ T cell hybridoma cells per well, in DMEM containing 10% FCS and 5 mM or 25 mM glucose (low or high glucose media, respectively). After 24 hours, the supernatants were removed and the production of IL-2 was measured by culturing supernatants in the presence of the IL-2-dependent cell line, CTLL-2. Proliferation of CTLL-2 cells was measured by the incorporation of ³[H] thymidine. For the antigen presentation assays, the 8F10 and IIT3 T cell hybridomas directed to the insulin B:12-20, and B:13-21 epitopes, respectively, were used (7).

Histology and Imaging.

Microscopy imaging was performed using an Eclipse E800 microscope (Nikon, Tokyo, Japan) equipped with CFI Plan Apo Lambda DM 20× air objective, X-Cite 120PC light source (Excelitas Technologies), EXi blue fluorescence microscopy camera, and QCAPTURE 64-bit v2.9.13 acquisition software (QImaging).

Islets, Pancreatic Stroma, and Lymph Node Isolation.

Pancreata were perfused through the common bile duct with 5.0 mL of calcium-free HBSS supplemented with 400.0 μg/mL of collagenase. Pancreata were then removed, and digested in a 37° C. water bath followed by vigorous shaking for 90 seconds, washed three times in HBSS, and then passed through a 70.0-μm strainer to retain the islets. Cells that passed the through the strainer represented the pancreatic stroma and were filtered a second time through a 40.0-μm filter to prepare a single-cell suspension. Islets retained on the 70.0-μm filter were then flushed into a Petri dish for hand-picking using a zinc-chelating dye, Dithizone (200 μg/mL 10% DMSO PBS; Sigma), to identify the islets. Hand-picked islets were then dispersed using Cell Dissociation Solution Non-Enzymatic (Sigma) for 10 minutes at 37° C. Lymph nodes and spleens were digested by incubation at 37° C. in DMEM supplemented with 10% fetal calf-serum, 125 μg/mL Liberase TL (Roche Life Science, Indianapolis, Ind.), and 50 μg/mL DNase I (Roche Life Science). All single-cell suspensions were then incubated with 2.4G2 conditioned media (PBS, 1% BSA, and 50% 2.4G2 in DMEM) at 4° C. for 15 minutes to block FC receptors. Samples were then stained with fluorescent antibodies for flow cytometry or sorting.

Antibodies for Flow Cytometry and Sorting.

Flow cytometry data were acquired on a FACSCanto II (BD Biosciences, San Jose, Calif.) and analyzed on FLOWJO v10.2 software (FlowJo, LLC, Ashland, Oreg.). Cell sorting was performed using a FACSAria II (BD Biosciences). The following antibodies were purchased from BioLegend: BV510 anti-CD45 (30-F11), Pacific Blue (PB) anti-I-A/I-E (pan MHC-II), FITC anti-CD3 (2C11), FITC and APC anti-F4/80 (BM8), PE-Cy7 anti-CD11 b (M1/70), PE anti-CD103 (2E7), PerCP-Cy5.5 anti-Ly6C (AL-21), PerCP-Cy5.5 anti-B220 (RA3-6B2), and APC anti-CD64 (X54-5/7/1). APC-eFluor780 anti-CD11c (N418), PerCP-eFluor710 anti-CD8 (53-6.7), eFluor450 anti-Ki-67 (SolA15), and APC FoxP3 (FJK-16) were purchased from eBioscience (Waltham, Mass.). For intracellular staining, the FoxP3 Fix/Perm kit (eBioscience) was used according to the manufacturer's instructions.

### Results

### Evaluation of C57BL/6 Mice

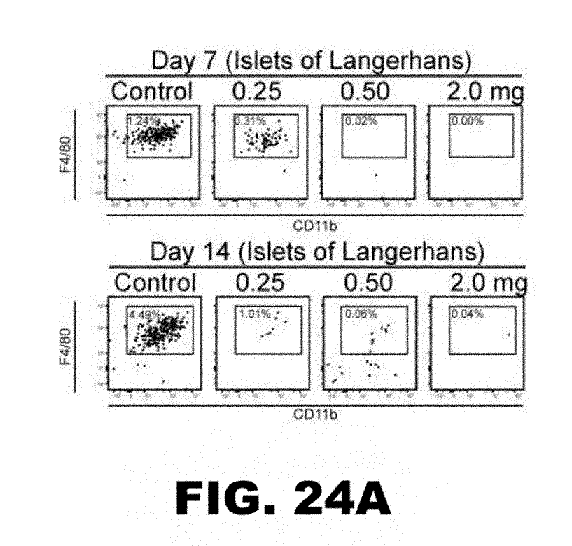

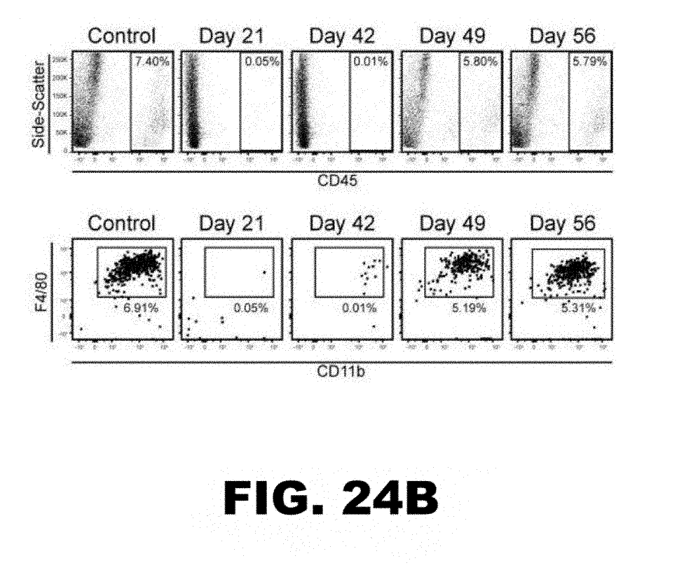

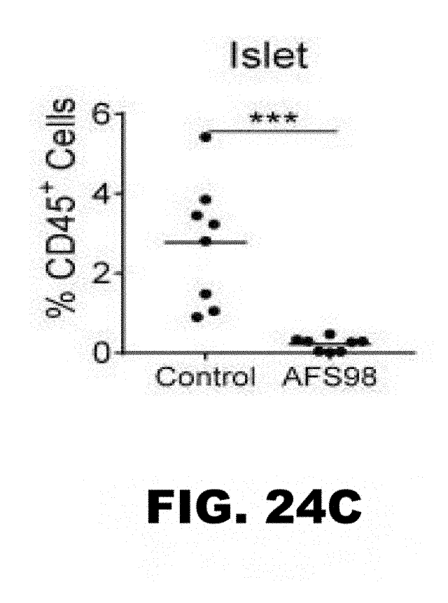

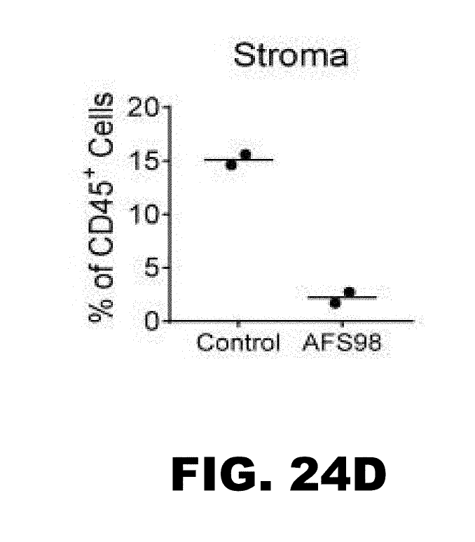

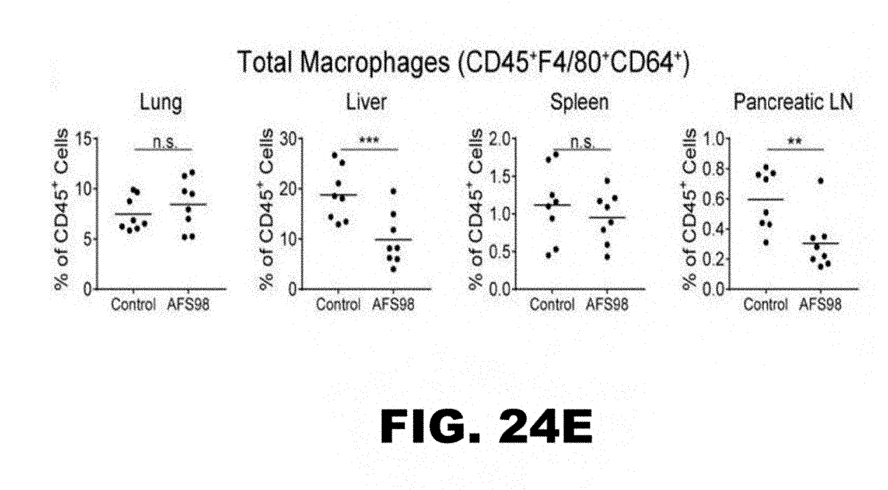

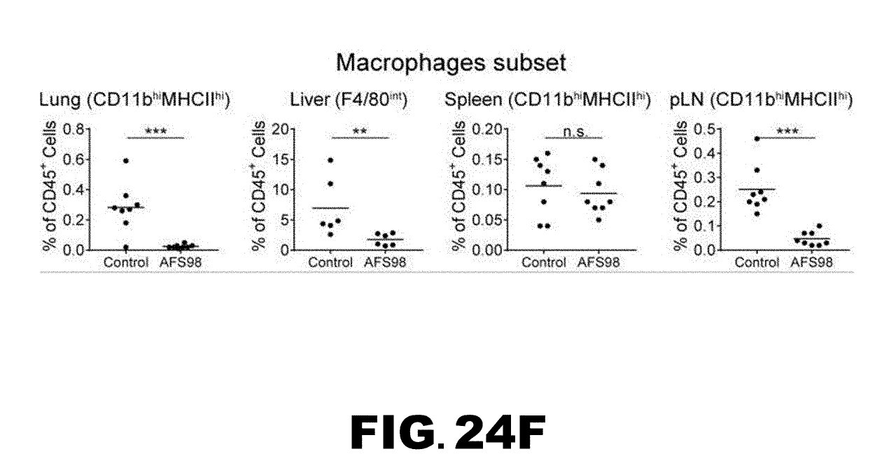

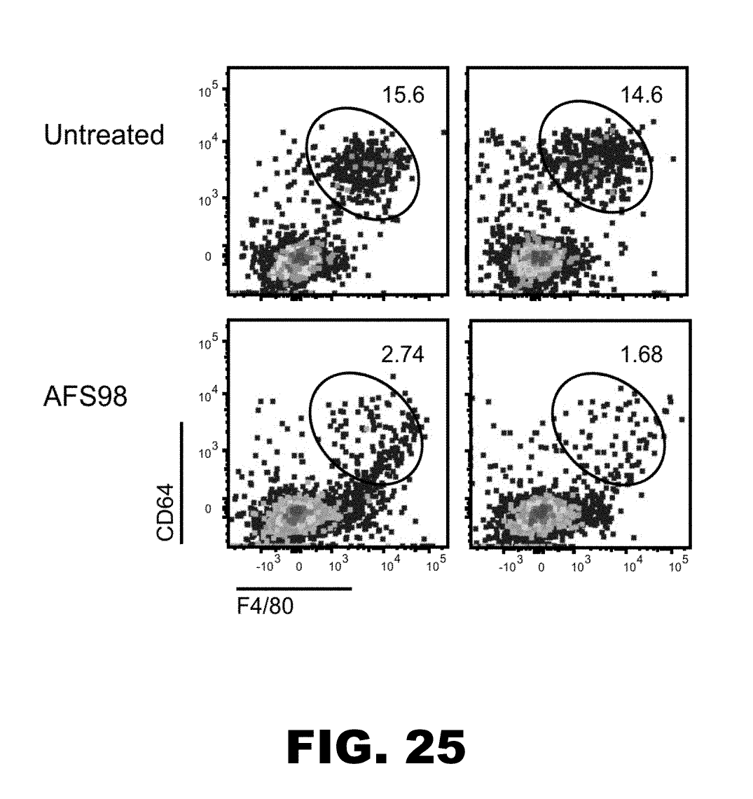

First, the nondiabetic B6 strain, following treatment with the monoclonal antibody against CSF-1R was evaluated. Injecting B6 mice with 0.25, 0.50, and 2.0 mg of AFS98 led to a dose-dependent elimination of the intraislet macrophages (FIG. 24A). This reduction was observed as early as 7 days after treatment and was equally evident at day 14. The depletion was long lived. A 2.0-mg dose of AFS98 resulted in elimination of the islet macrophage lasting for at least 6 weeks (FIG. 24B). Macrophages started to return to the islets by 6 weeks and were at normal levels by 7 weeks after antibody treatment. The depletion of macrophages in the islets was consistent over multiple experiments (FIG. 24C). The macrophages of the stroma were also affected by the AFS98 treatment. The macrophage reduction was almost complete resulting in an ˜85% reduction compared with control-treated mice (FIG. 24D and FIG. 25).

In addition to islets, several other tissues were examined. In most, a set of the resident macrophages was also affected 7-14 days after antibody administration. Total macrophages by expression of F4/80 and CD64 were analyzed and calculated their reduction as shown in FIG. 24E. However, it was evident that the sensitivity to AFS98 treatment was variable (FIG. 24F). In the lung, alveolar macrophages were not affected, yet the CD11b⁺ interstitial macrophages were depleted (15) (FIG. 24E and FIG. 24F). In the liver, there are two macrophage populations defined by expression of F4/80. The F4/80^{hi} Kupffer cells were reduced by about 50% (FIG. 24E and FIG. 24F). The F4/80^{int} monocyte-derived macrophages were almost completely depleted by AFS98 treatment. This last finding suggest that these macrophages most likely represent the recently identified liver capsular macrophages (16). The spleen macrophages were not affected (FIG. 24E and FIG. 24F). However, in the pancreatic lymph node, a set of macrophages characterized by CD11 b (similar in surface marker expression to the lung interstitial macrophages) were >80% reduced. Thus, not all tissue macrophages are as sensitive to anti-CSF-1R as the ones in the islets.

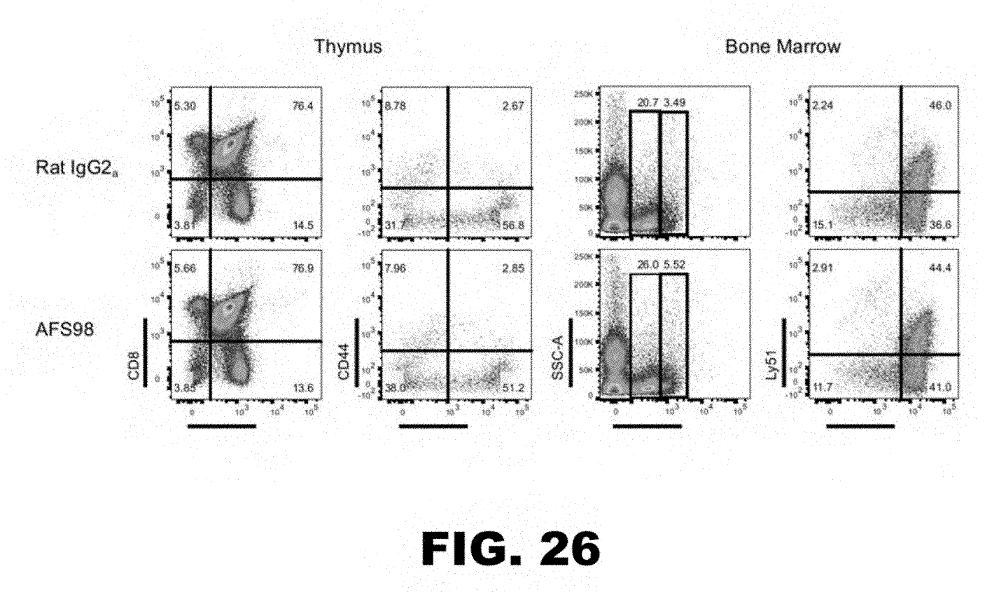

In summary, treatment with AFS98 led to variable effects on the macrophages residing in various tissues, while in islets, the effects were pronounced and prolonged. AFS98 treatment was also evaluated whether it could affect the number of T and B cells and their differentiation in thymus and bone marrow. No impairment in their numbers and differentiation patterns in thymus or bone marrow was found (FIG. 26).

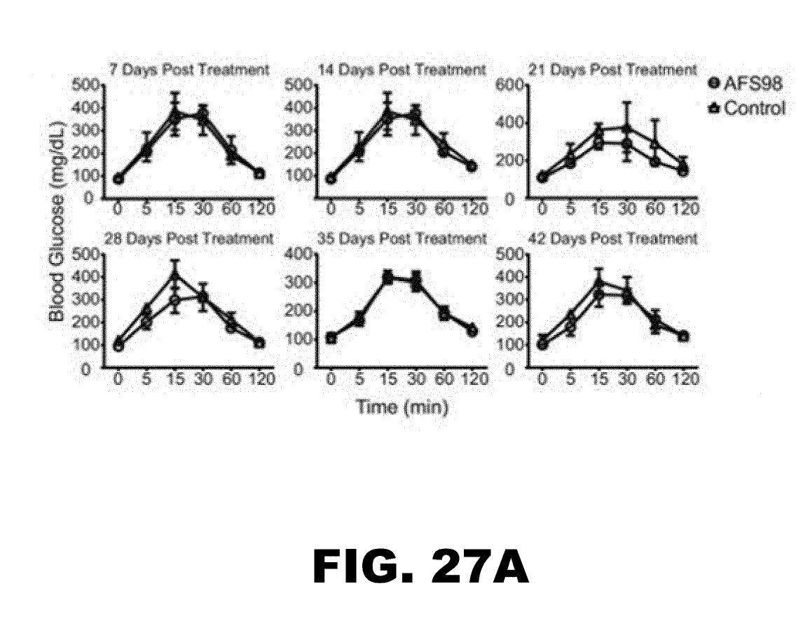

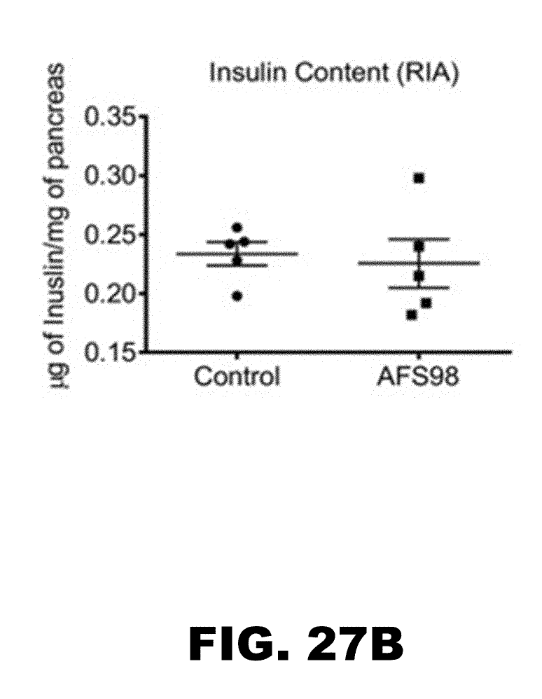

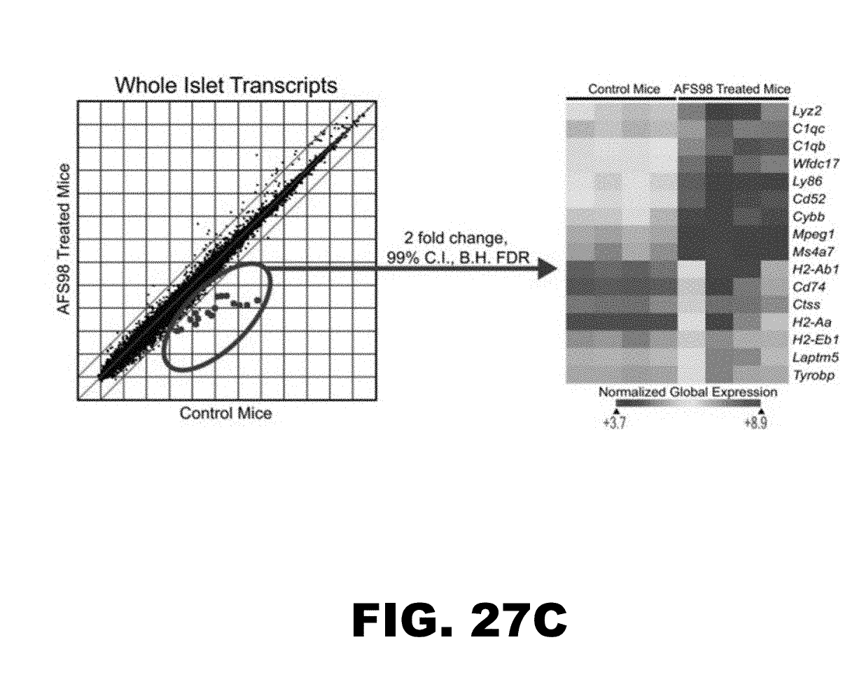

As seen in the op/op mouse, the absence of macrophages from birth can lead to systemic disruption of mouse homeostasis. Therefore, the two basic parameters of islet function after macrophage depletion: glucose tolerance and beta-cell insulin content were evaluated. The glucose tolerance test measures the ability of the beta cell to sensor and respond to glucose, release insulin, and return the mouse to euglycemia. Following treatment of the B6 mice with the AFS98 antibody, neither glucose tolerance nor insulin content was affected (FIG. 27A and FIG. 27B). Glucose tolerance was unimpaired even after 6 weeks of macrophage depletion in the islets (FIG. 27A). Measured pancreatic insulin content was stable at 3 weeks after depletion (FIG. 27B).

A more global and unbiased measure of islet health is to evaluate the whole transcriptome. B6 mice were treated with 2.0 mg of AFS98 or control IgG2ₐ at 3 weeks of age, and whole islets were isolated at 6 weeks of age. Comparison of AFS98 versus IgG2ₐ-treated mice revealed 16 differentially expressed transcripts at a twofold change and 99% confidence interval in whole islets (FIG. 27C). These 16 changes were in transcripts known to be encoded strictly by the macrophage. These included transcripts previously reported to be up-regulated during diabetes progression (9). Therefore, the majority of the islet transcriptome was not significantly affected by macrophage depletion, and the only change was the loss of the islet macrophage. In conclusion, homeostasis of islet function was not affected in a detectable manner when macrophages were depleted several weeks after birth.

### Evaluation of NOD Mice.

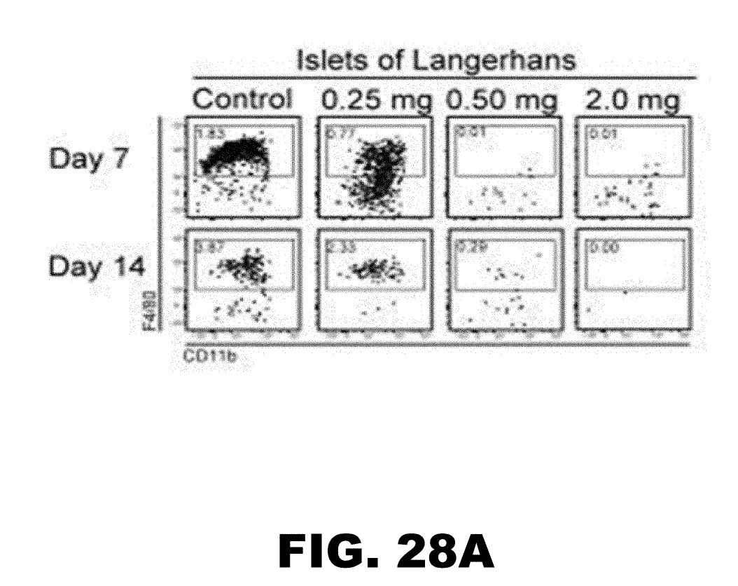

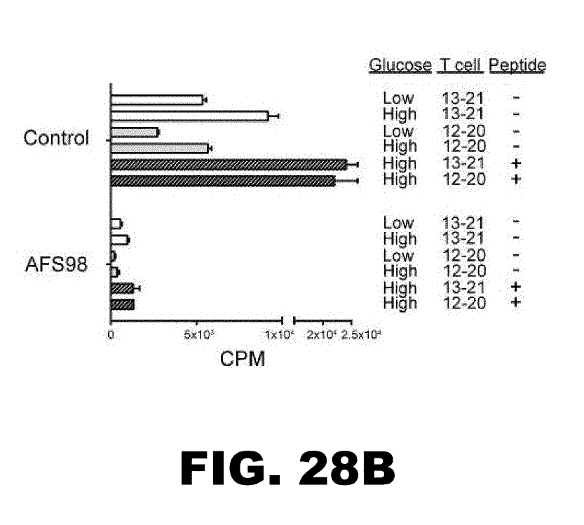

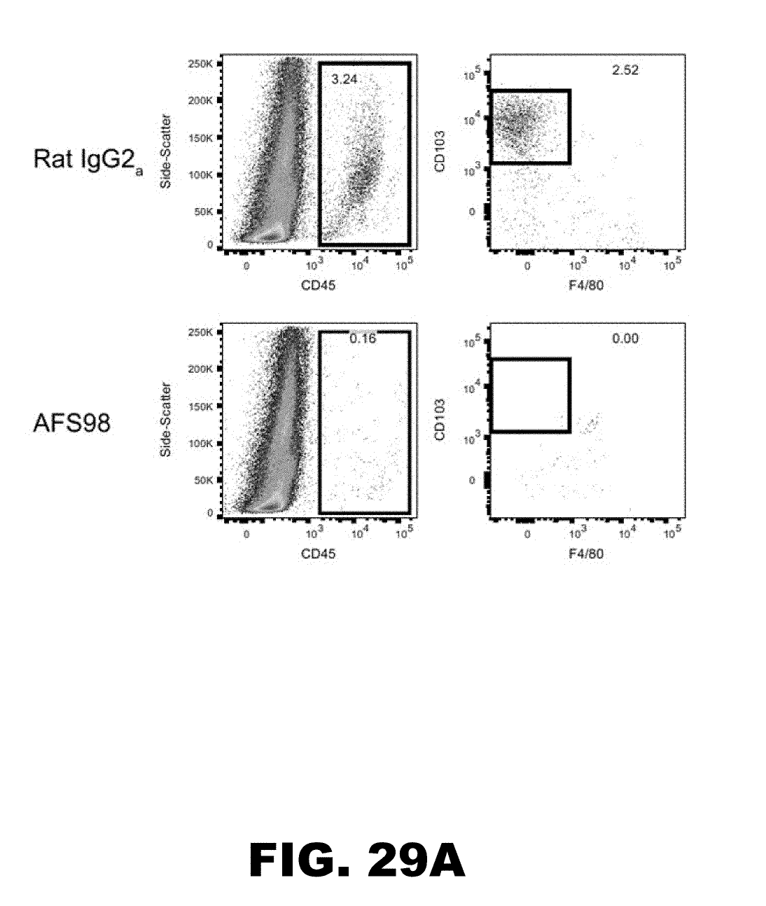

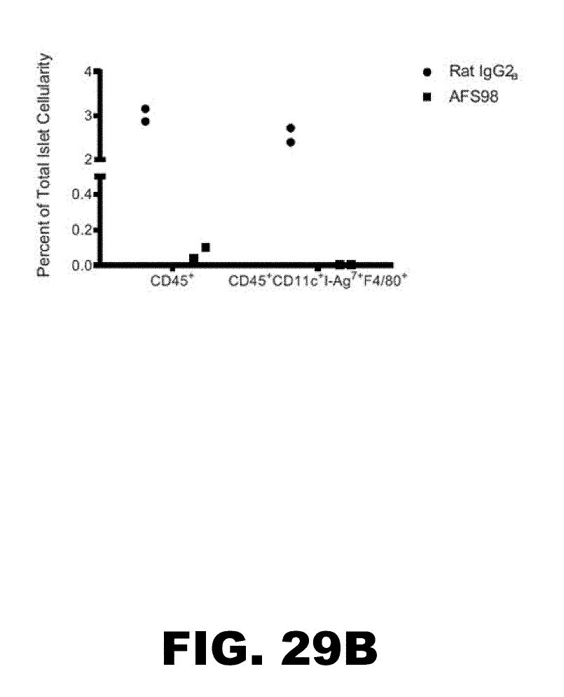

Islets. Similar to the results observed in B6 mice, FIG. 28A shows that treatment with 0.5 or 2.0 mg of AFS98 depleted the islet macrophages in 4- to 5-week-old NOD mice. Comparable results were obtained following treatment of NOD. Rag1^{-/-} mice with the AFS98 antibody (FIG. 29A and FIG. 29B). An analysis of NOD mice at 3-4 weeks of age showed that the initial islet infiltrating T cells were all CD4⁺ and mostly in contact with the intraislet macrophage (9). The islet macrophages express CD11 b, CD11c, and MHC-II highly on their surface. At this 3-4 week of age period, there are very few XCR1⁺ DCs in islets (10).

The colocalization of CD4 T cells with intraislet macrophages was confirmed by examining islets using two-photon microscopy. NOD mouse islets were examined at 3-4 weeks of age, the earliest age where one can identify the initial infiltrating T cells. Indeed, 27% of NOD islets had CD4 T cells, 70% of which were in contact with the F4/80⁺ macrophages, confirming our initial studies. In the mice that were injected with 0.5 mg of anti-CSF-1R at 2 weeks of age, the islets did not harbor any myeloid cells at 4 weeks of age. In these mice, only 3% of islets had a detectable CD4 T cell.

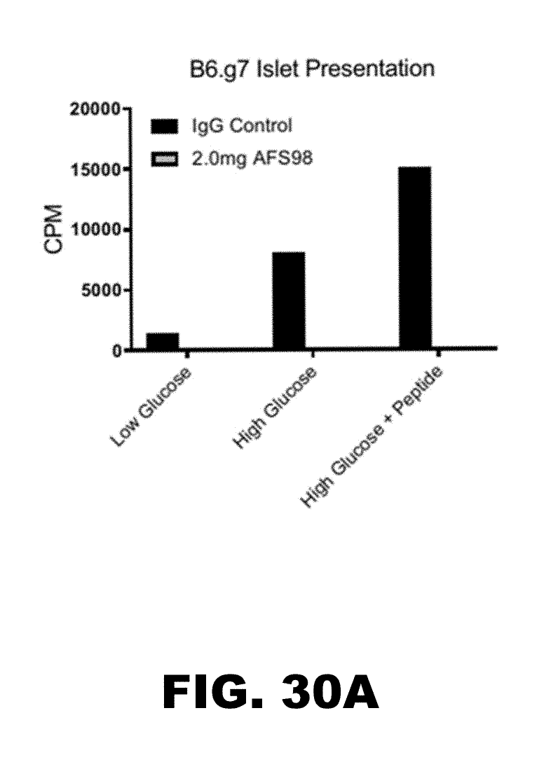

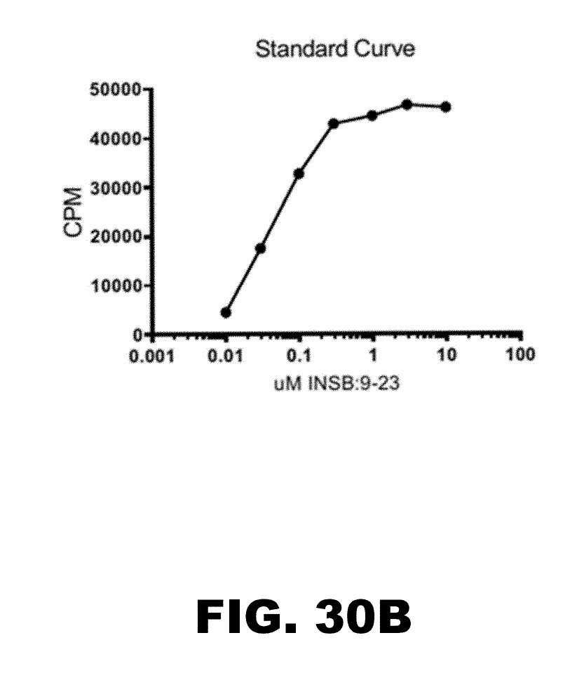

Next, insulin peptide presentation by isolating the islets, dispersing the cells, and culturing them with insulin-reactive CD4 T cell hybridomas was examined. Depletion of islet macrophages resulted in marked reduction of presentation to two different CD4 T cell hybridomas recognizing different MHC-II epitopes of insulin (17). The addition of the cognate peptides reflects the availability of MHC-II⁺ presenting cells. As noted in FIG. 28B, this addition did not lead to presentation in the islets of the treated mice, reflecting the paucity of presenting cells and the inability of any other islet cell to express MHC-II or present peptide. The same findings were reproduced in B6.g7 mice (FIG. 30A and FIG. 30B).

Lymph Node Responses.

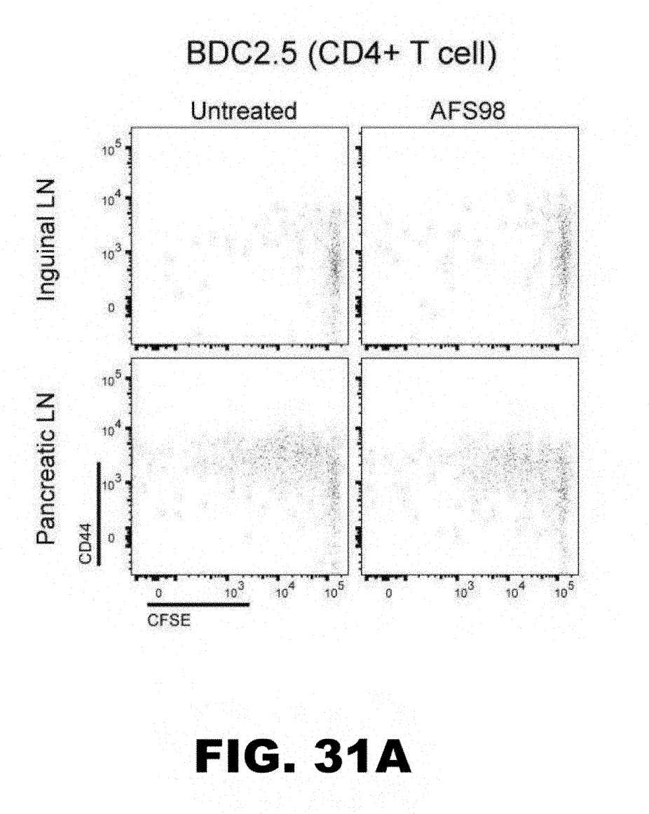

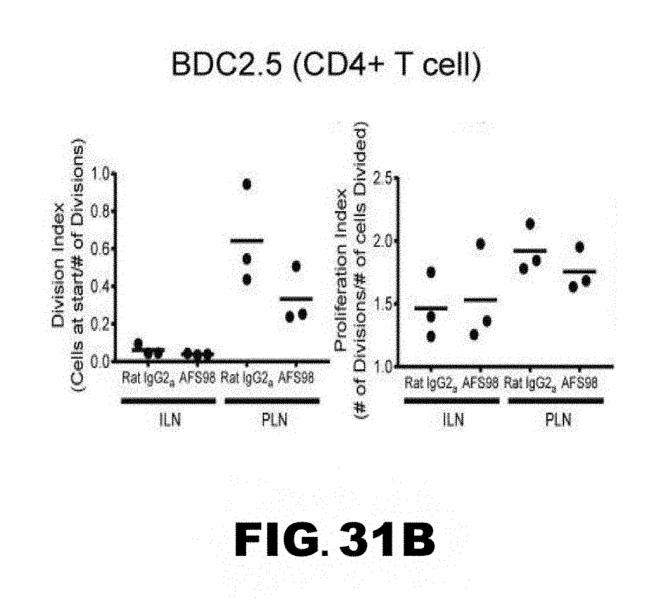

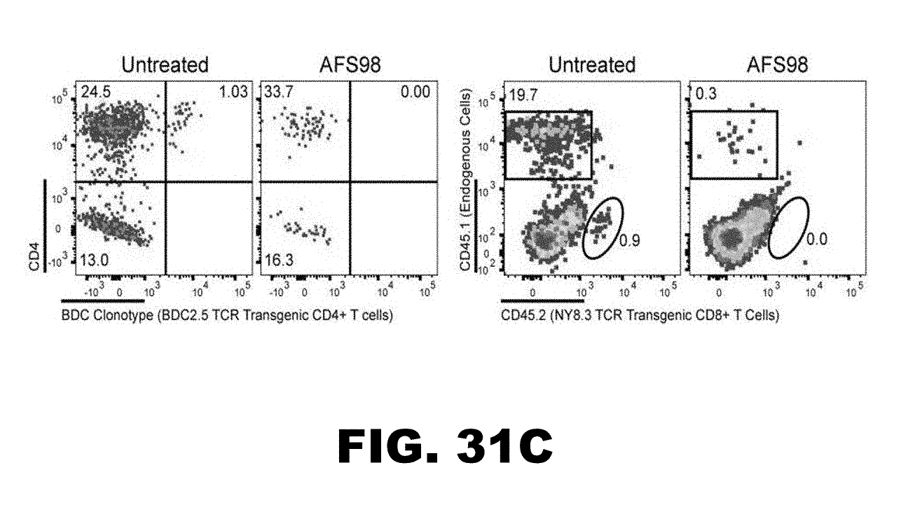

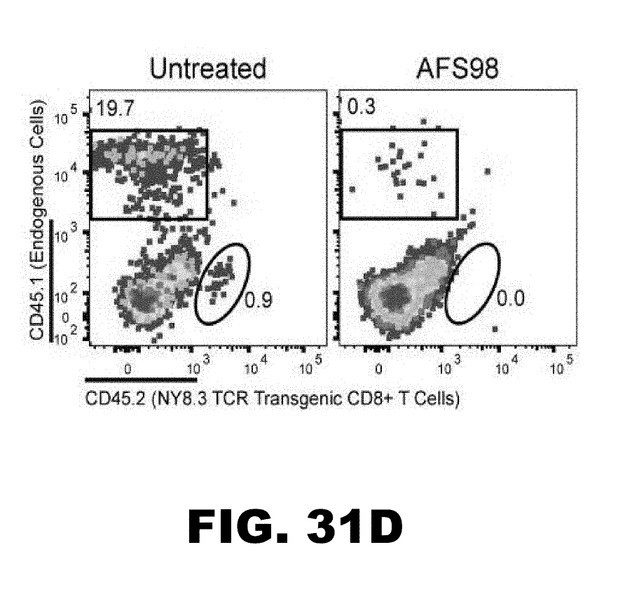

While the islets from the macrophage-depleted mice were impaired in antigen presentation, this was not the case in the peripheral lymph nodes. This was determined using two approaches: carboxyfluorescein succinimidyl ester (CFSE) dilution of transferred T cells and recall response by ELISpot. For CFSE dilution experiments, CD4 (BDC2.5) or CD8 (NY8.3) T cell clones were transferred into control or AFS98-treated NOD. BDC2.5 responds to a chromogranin peptide in the context of I-A^{g7} (18), while the NY8.3 T cell divides in response to a peptide derived from IGRP, the islet-specific glucose-6-phosphatase-dependent catalytic subunit-related protein, in the context of H-2Kd (19, 20). FIG. 31A and FIG. 31B shows that both T cell clones proliferated in the draining pancreatic lymph node, but not in the distant inguinal lymph node. Next, to determine if depletion of the islet macrophage affected the trafficking of diabetogenic cells to the islets, the entrance of TCR transgenic T cells into them was examined. Both BDC2.5 and NY8.3 T cells entered islets of control mice but neither entered the islets from AFS98-treated mice (FIG. 31C). While the T cells entered and reacted to antigen presented in the pancreatic lymph node, this was not the case in the islets.

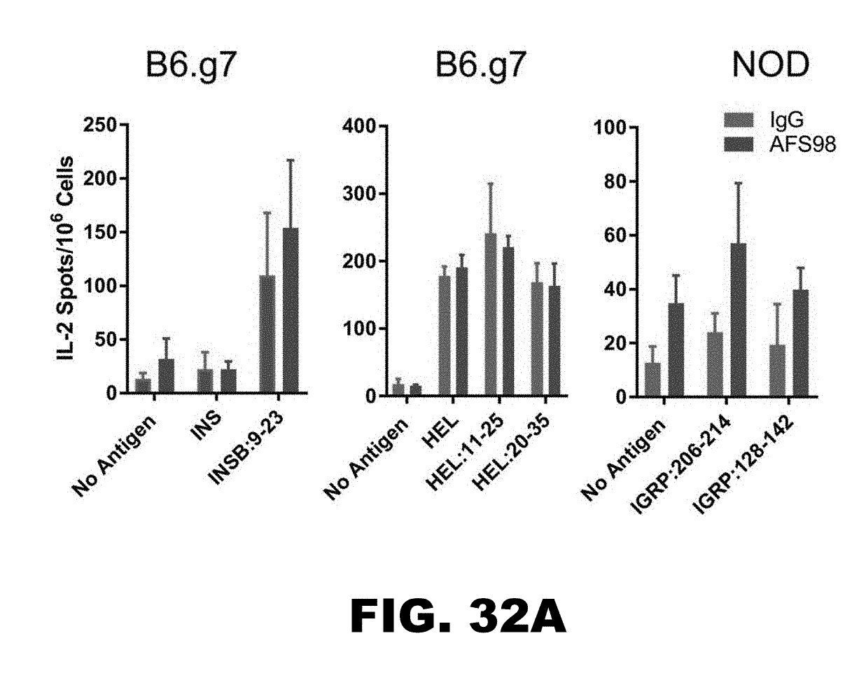

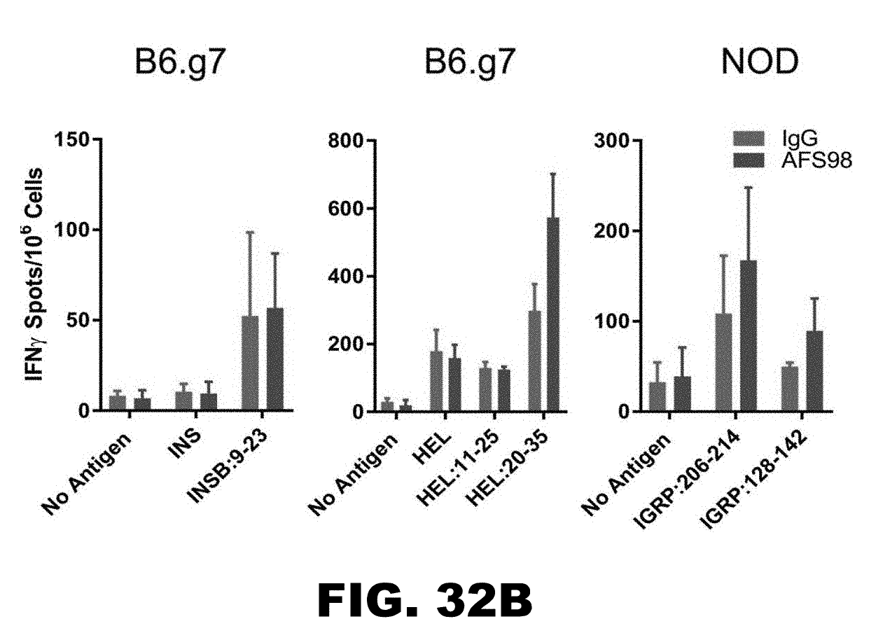

In the second approach, AFS-treated mice were immunized with various autoreactive peptides in the footpads and the T cell response was tested a week later; there was no impairment of the response (FIG. 32A and FIG. 32B). Immunization with the insulin B:9-23 peptide elicited an IL-2 and IFN-γ response to the peptide but not to the insulin protein, as reported before (17). Immunization with peptides from IGRP known to elicit CD4 or CD8 T cell responses was also unaffected by AFS98 treatment. The CD4 or CD8 T cell responses to the foreign protein hen egg lysozyme (HEL) were also unaffected.

In brief, examination by flow cytometry, direct microscopy of islets, antigen presentation assays, and T cell migration assays shows that the lack of macrophages translates into an absence of early CD4 T cell infiltration and antigen presentation capability of the islets. In contrast, the lymph nodes in the AFS-treated mice were active in antigen presentation.

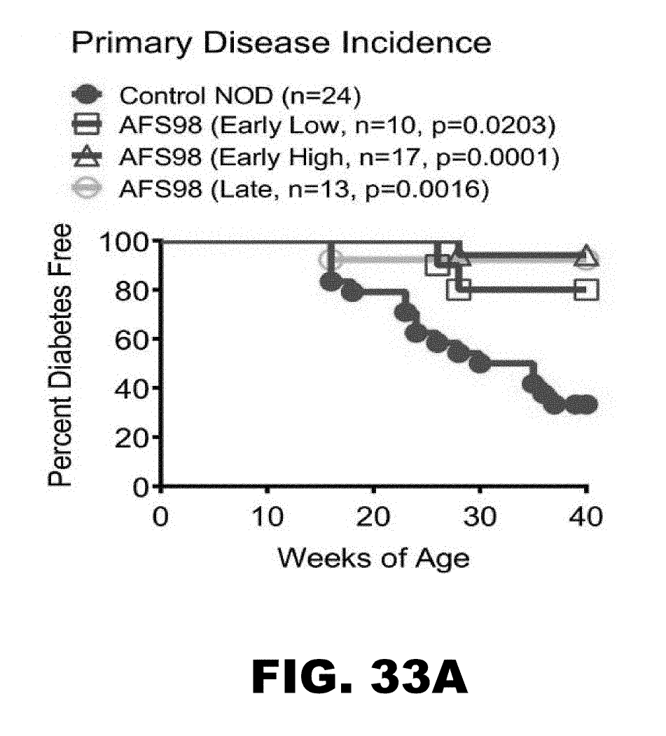

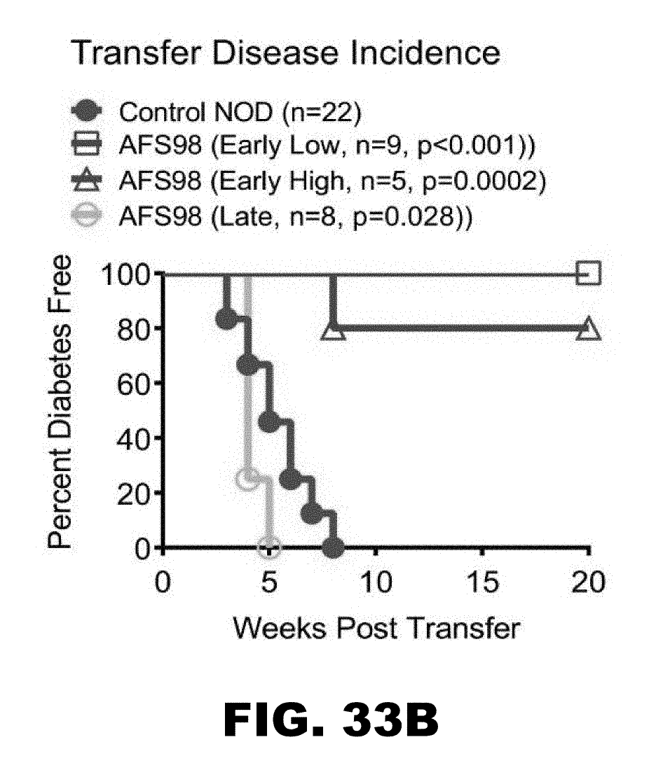

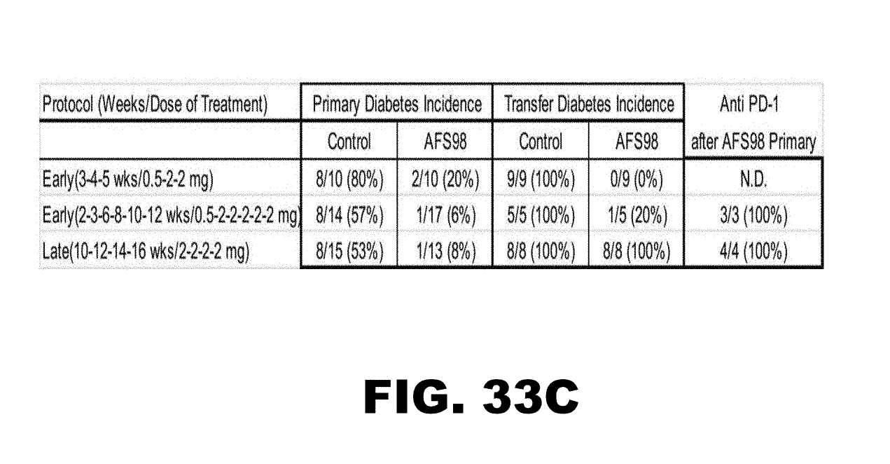

Diabetes. Female NOD mice were followed for diabetes after treatment with AFS98 or control rat IgG2ₐ antibody. In two experiments, mice were treated at 2-3 weeks of age, a time when the diabetogenic process is starting in limited islets. The treatment was continued for several weeks using two different concentrations of antibody. In a third experiment, NOD mice were treated starting at 10 weeks of age, a time when the diabetogenic process is active and most islets are already infiltrated by both CD4 and CD8 T cells. Both treatments led to a marked reduction in diabetes incidence when the mice were followed for 40 weeks. The pooled results are shown in FIG. 33A. FIG. 33C shows the results of the individual experiments. Combining the three experiments, 4 of 40 AFS98-treated mice and 24 of 39 of the control mice became diabetic.

At the end of the 40-42 weeks of observation, two manipulations were performed on the AFS98-treated mice. First, the mice were administered an anti-PD-1 monoclonal antibody and this led to the rapid development of diabetes in 7 of 7 of the mice evaluated (FIG. 33C). Others have found a profound regulatory effect of the PD-1/PD-L1 pathways in regular diabetogenesis, and at least this pathway is operational following AFS98 treatment (21-23). Anatomically, insulin-reactive T cells express PD-1, while in islets the beta cells, vascular endothelium, macrophages, and inflammatory DCs express high levels of PD-L1 (21, 24, 25).

Second, splenocytes from the AFS98-treated mice were transferred into NOD.Rag1^{-/-} mice. NOD splenocytes transfer disease to normally nondiabetic NOD.Rag1^{-/-} mice and is used as a measure of the diabetogenic potential of the T cells in the NOD mouse following genetic or pharmaceutical manipulations. The mice first treated with AFS98 at the 2- to 3-week period transferred diabetes poorly; only 1 of 14 became diabetic 20 weeks after transfer (FIG. 33B and FIG. 33C). In contrast, splenocytes from 10-week, AFS98-treated NOD mice transferred diabetes to 8 of 8 recipients.

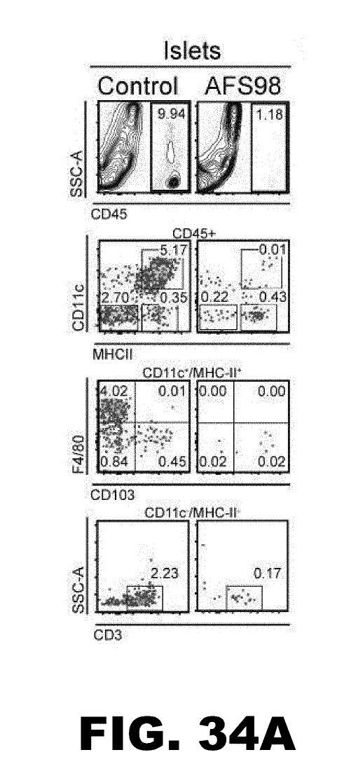

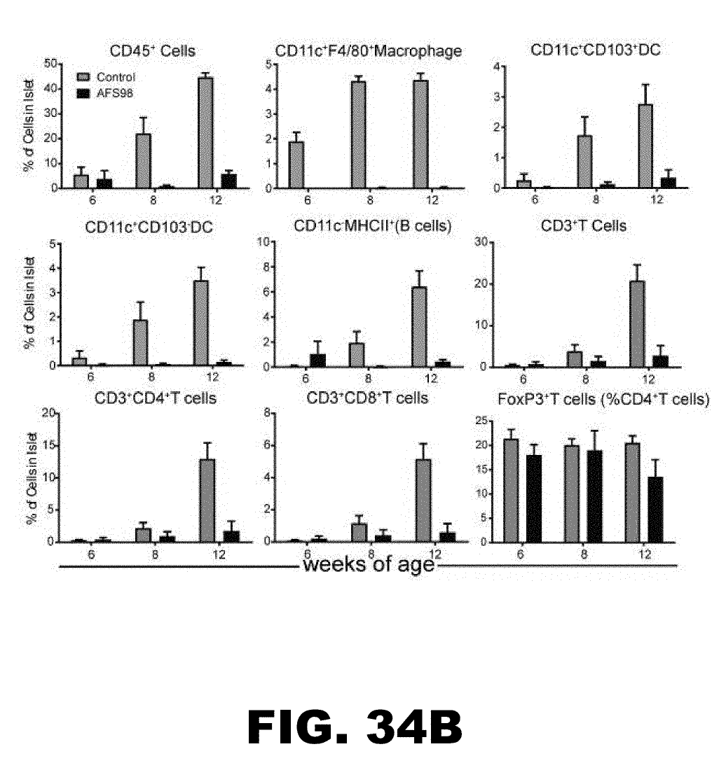

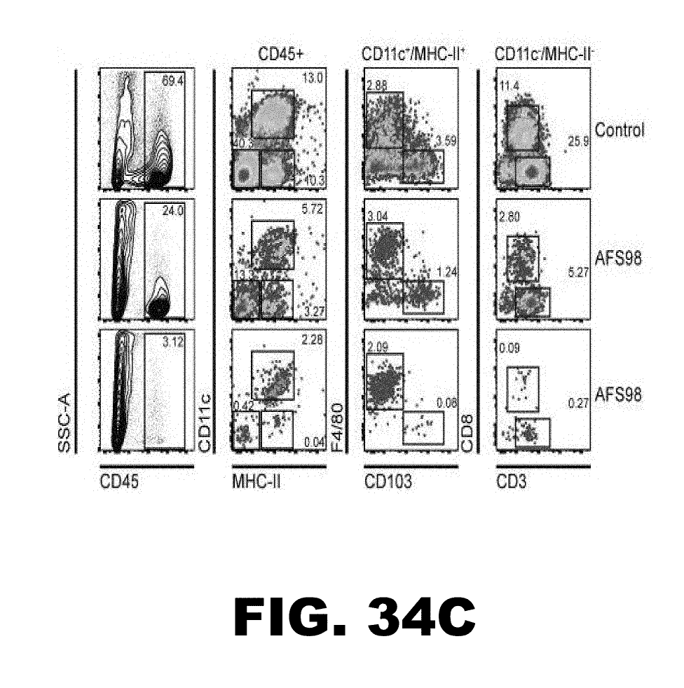

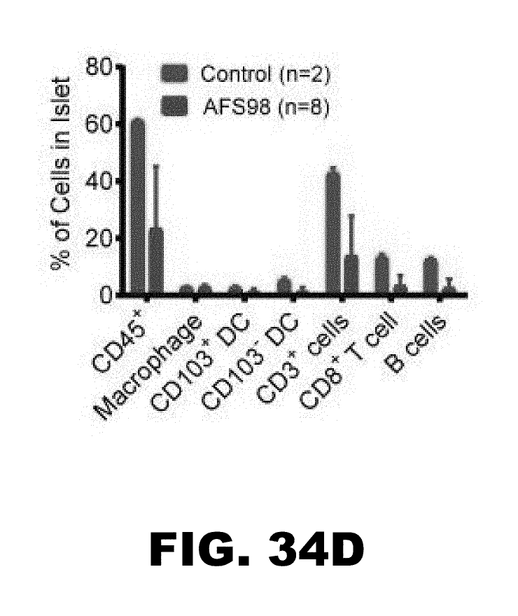

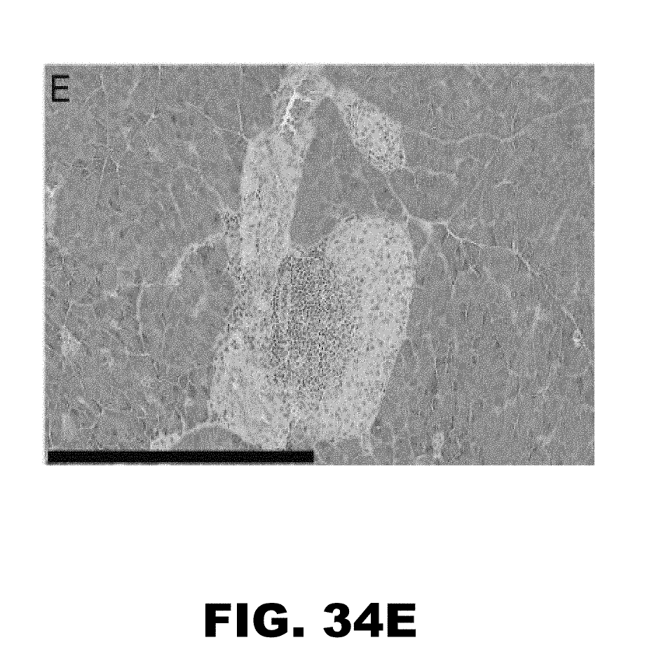

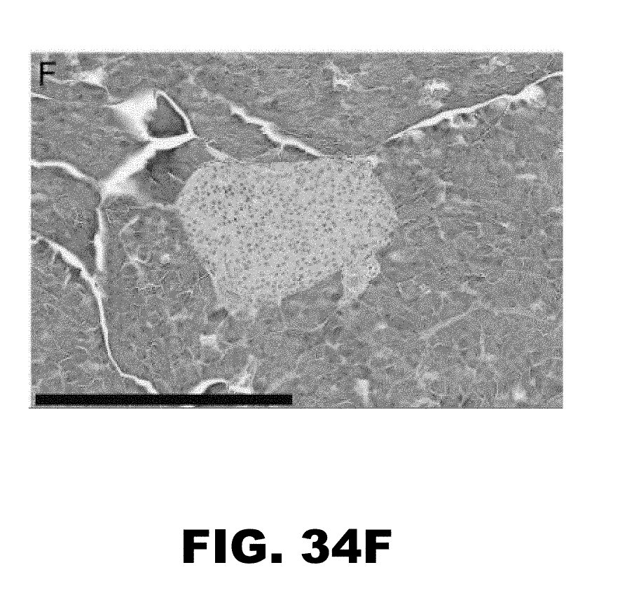

Islets were also examined for their leukocyte content at various time points after their treatment with AFS98. FIG. 34A, FIG. 34B, and FIG. 34C, and FIG. 34D show results from mice treated at 2-3 weeks of age. AFS98 treatment resulted in a reduction of total leukocytes (CD45⁺) from ˜10% in controls to ˜1% in the treated mice by 8 weeks after treatment (FIG. 34A). There was also a reduction of F4/80⁺ macrophages from ˜4% of total islet cellularity to undetectable levels. Over time, the control NOD mice showed progressive increase in the CD45⁺ cells (FIG. 34B). After the end of the observation period—at 44 weeks—a number of the islets from AFS-treated mice were infiltrated. Two phenotypes: 4 of 8 mice contained ˜20-25% CD45⁺ cells in the islets, and 4 of 8 mice contained 2-5% CD45⁺ cells in the islets were observed (FIG. 34C and FIG. 34D). (The IgG-treated control NOD mice contained ˜60-70% CD45⁺ cells in islets.) Corroborating the flow cytometry ˜50% of the pancreata examined had a degree of peri-insulitis (FIG. 34E). However, the other 50% of pancreata examined showed little to no insulitis (FIG. 34F). Therefore, macrophage depletion is protective in NOD mice long term, despite the infiltration or expansion of leukocytes in ˜50% of the mice.

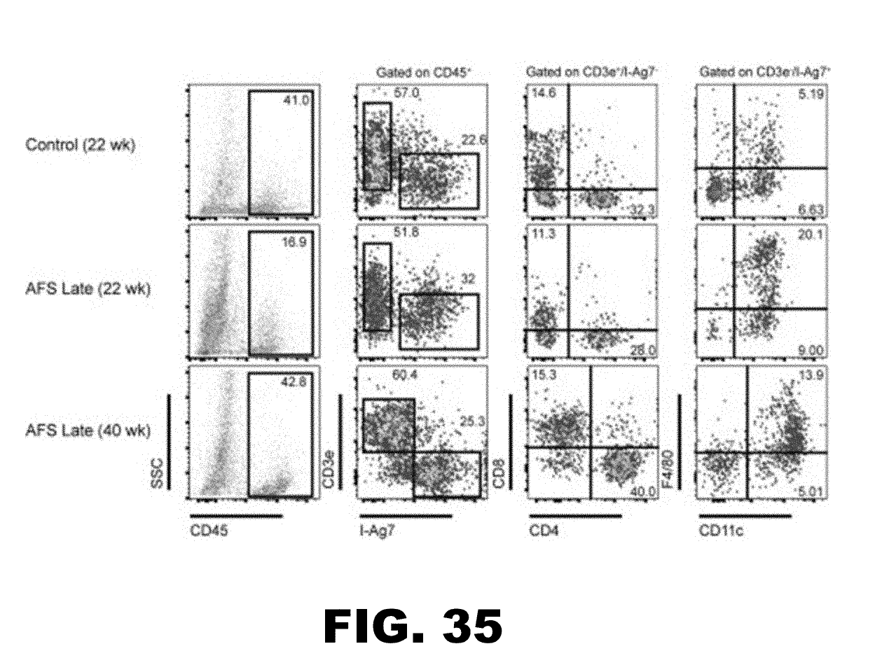

In mice treated with AFS98 starting at 10 weeks of age, there also was a reduction in the number of leukocytes in islets at 22 weeks of age. However, by 40 weeks of age, all of the mice had comparable infiltrates to a 22-week-old NOD mouse (FIG. 35). Thus, late macrophage depletion does not stop the infiltration of islets, despite preventing the development of hyperglycemia.

These results suggest that macrophage depletion can act before and after the diabetogenic T cell pool in the NOD has fully developed. Early AFS98 treatment blocks diabetes by eliminating a key antigen-presenting cell in islets, preventing early entry of activated T cells, and limiting the expansion of the diabetogenic T cells. In contrast, in late-treated NOD mice, there are active diabetogenic T cells but disease is controlled by the development of a regulatory condition through at least one mechanism, PD-1/PD-L1 interaction. In both situations, islet infiltration with preservation of islet function was observed.

### Discussion

Injection of NOD mice with antibodies directed against CSF-1R depleted the islet resident macrophages while having a variable effect on macrophages of secondary lymphoid tissues and various other organs. The treatment had a profound effect in the development of diabetes. The manner in which the islet macrophage was eliminated by the antibody treatment is not clear, although it does not involve an acute inflammatory reaction (14). The islet transcriptome analysis displayed no up-regulation of inflammatory transcripts following macrophage depletion (FIG. 27C). CSF-1R signaling is crucial for sustaining macrophage viability, which is most likely the reason for its loss following the antibody treatment (11).

There are two distinct scenarios that likely contributed to the attenuation of the active autoimmune process: (i) the impairment of islet antigen presenting function and (ii) the development of a regulatory process. Concerning the islets, these studies confirm the centrality of the resident macrophage in the pathophysiology of pancreatic islets. The islet macrophage arises during embryonal development. It is required for the early development of islets and during postnatal growth as is evident in the op/op mouse that lack CSF-1 (4, 5). However, in adult mice, islet function is not impaired when the macrophage is absent (our studies in B6 mice). The islet macrophage is highly activated, as evidenced by their expression of TNF and IL-1 transcript and protein, and high expression levels of MHC-II (5, 10). In the context of autoimmunity, such as in NOD mice, autoreactive T cells escape thymic control and enter islets whereupon they establish long-lived contact with the macrophage (26).

The macrophage-depleted islet per se was impaired in its capacity to receive diabetogenic T cells. In their absence, lymphocyte entry into islets was impaired. The immunofluorescence and two-photon imaging of isolated islets showed the majority of CD4 T cells in islets in contact with the macrophage. Indeed, previous analyses showed macrophage filipodia extending into the blood vessel lumens (8). I.V. injecting 0.5-μm latex beads coated with antibodies to I-A^{g7} localized the beads to the macrophage/blood vessel interface. These findings point to the macrophage filopodia as the anatomical element that captures the diabetogenic CD4 T cells, while the high-expression MHC-II may well be the key molecular substrate (8). In sum, the present and past studies point to the macrophages as the beacon, the true gatekeeper for entrance of lymphocytes into the islet.

Three pointed findings followed the initial depletion of macrophages. First, the effect of AFS98 treatment applied to mice already undergoing an active diabetogenic process. At 10 weeks of age, the autoimmune process is progressing; our own studies showed a striking inflammatory signal at this time. However, AFS98 treatment at this time was effective. This indicates that AFS98 inhibits when there are active diabetogenic T cells. Thus, the inhibitory effect of macrophage depletion is not necessarily accounted for by a lack of T cell priming. Second is that after the period of macrophage depletion, the islet becomes progressively receptive to the entrance of T cells and new macrophages. This was evident when examining the islets by flow cytometry. Despite this renewed accumulation of leukocytes into the islets, the beta cells were preserved. Histological examination at the 42-week period showed the preservation of beta cells, albeit many islets showed the peri-insulitic lesion next to otherwise normal beta cells. This finding suggests that the newly arriving monocyte-derived macrophages or other cells in islets or in lymph nodes are not driving forward the process and may participate by controlling it. For example, the incoming macrophages may be inhibitory in a way akin of what happens in the tumor environment. Finally, a regulatory process involving the PD-1 and PD-L1 interacting molecules was evident. The mice protected by AFS98 until over 40 weeks of age developed diabetes if given anti-PD-1. At face value, this result tells us that the active process had been under checkpoint control following the initial macrophage deletion.

Future studies should examine the site of the control pathway, whether it is the lymph nodes or the islets, many of which had the peri-insulitic lesion. In NOD diabetogenicity, peripheral lymph nodes play a key role in the early program of the autoimmunity (27-29). It is not shown here that the lymph nodes after AFS treatment could be an effective site of presentation of either diabetogenic or foreign antigens. It is not because of the lack of presenting function in lymph nodes that diabetes is curtailed. However, the nodes could be the anatomical substrate of the regulatory process involving the PD-1/PD-L1 pathway. The lymph nodes are depleted of their resident macrophages, albeit not to the extent as islets. Thus, the presentation that results in the activation of the PD-1/PD-L1 activation could be caused by DC or B cells. Lymph node macrophages at face value would foster positive interactions in a normal situation but, in their absence, would drive the process into a regulatory pathway.

Various inflammatory processes have been studied with variable results using antibodies to transiently remove tissue macrophages (30-35). Other treatment modalities have targeted blood monocytes. Clodronate-liposome treatment depletes circulating monocytes and affected a number of inflammatory reactions (36). These treatments over repeated periods of time resulted in the reduction in diabetes in NOD mice (36-40). In all of these instances, the reduction resulted in the loss of inflammatory macrophages, i.e., macrophages derived from monocytes that participated as effectors in the diabetogenic process. A study by Calderon et al. (41) showed that resident macrophages were not affected by injections of clodronate liposomes, although diabetes induced by the transfer of the activated BDC2.5 CD4⁺ T cells was affected. Diabetes induced by such transfer required the presence of new macrophages as effector cells modulated by IFN-γ production from activated T cells (42).

### REFERENCES

1. Perdiguero E G, Geissmann F. The development and maintenance of resident macrophages. Nat Immunol. 2016; 17:2-8.
2. Mass E, et al. Specification of tissue-resident macrophages during organogenesis. Science. 2016; 353:aaf4238.
3. Wiktor-Jedrzejczak W, et al. Total absence of colony-stimulating factor 1 in the macrophage-deficient osteopetrotic (op/op) mouse. Proc Natl Acad Sci USA. 1990; 87:4828-4832.
4. Banaei-Bouchareb L, et al. Insulin cell mass is altered in Csf1op/Csf1op macrophage-deficient mice. J Leukoc Biol. 2004; 76:359-367.
5. Calderon B, et al. The pancreas anatomy conditions the origin and properties of resident macrophages. J Exp Med. 2015; 212:1497-1512.
6. Ferris S T, et al. The islet-resident macrophage is in an inflammatory state and senses microbial products in blood. J Exp Med. 2017; 214:2369-2385.
7. Vomund A N, et al. Beta cells transfer vesicles containing insulin to phagocytes for presentation to T cells. Proc Natl Acad Sci USA. 2015; 112:E5496-E5502.
8. Calderon B, Carrero J A, Miller M J, Unanue E R. Cellular and molecular events in the localization of diabetogenic T cells to islets of Langerhans. Proc Natl Acad Sci USA. 2011; 108:1561-1566.
9. Carrero J A, Calderon B, Towfic F, Artyomov M N, Unanue E R. Defining the transcriptional and cellular landscape of type 1 diabetes in the NOD mouse. PLoS One. 2013; 8:e59701.
10. Ferris S T, et al. A minor subset of Batf3-dependent antigen-presenting cells in islets of Langerhans is essential for the development of autoimmune diabetes. Immunity. 2014; 41:657-669.
11. Sudo T, et al. Functional hierarchy of c-kit and c-fms in intramarrow production of CFU-M. Oncogene. 1995; 11:2469-2476.
12. MacDonald K P A, et al. An antibody against the colony-stimulating factor 1 receptor depletes the resident subset of monocytes and tissue- and tumor-associated macrophages but does not inhibit inflammation. Blood. 2010; 116:3955-3963.
13. Sauter K A, et al. Pleiotropic effects of extended blockade of CSF1R signaling in adult mice. J Leukoc Biol. 2014; 96:265-274.
14. Hume D A, MacDonald K P A. Therapeutic applications of macrophage colony-stimulating factor-1 (CSF-1) and antagonists of CSF-1 receptor (CSF-1R) signaling. Blood. 2012; 119:1810-1820.
15. Kopf M, Schneider C, Nobs S P. The development and function of lung-resident macrophages and dendritic cells. Nat Immunol. 2015; 16:36-44.
16. Sierro F, et al. A liver capsular network of monocyte-derived macrophages restricts hepatic dissemination of intraperitoneal bacteria by neutrophil recruitment. Immunity. 2017; 47:374-388.e6.
17. Mohan J F, et al. Unique autoreactive T cells recognize insulin peptides generated within the islets of Langerhans in autoimmune diabetes. Nat Immunol. 2010; 11:350-354.
18. Delong T, et al. Diabetogenic T-cell clones recognize an altered peptide of chromogranin A. Diabetes. 2012; 61:3239-3246.
19. Lieberman S M, et al. Identification of the beta cell antigen targeted by a prevalent population of pathogenic CD8+ T cells in autoimmune diabetes. Proc Natl Acad Sci USA. 2003; 100:8384-8388.
20. Wang J, et al. In situ recognition of autoantigen as an essential gatekeeper in autoimmune CD8+ T cell inflammation. Proc Natl Acad Sci USA. 2010; 107:9317-9322.
21. Ansari M J I, et al. The programmed death-1 (PD-1) pathway regulates autoimmune diabetes in nonobese diabetic (NOD) mice. J Exp Med. 2003; 198:63-69.
22. Fife B T, et al. Insulin-induced remission in new-onset NOD mice is maintained by the PD-1-PD-L1 pathway. J Exp Med. 2006; 203:2737-2747.
23. Wang J, et al. Establishment of NOD-Pdcd1−/− mice as an efficient animal model of type I diabetes. Proc Natl Acad Sci USA. 2005; 102:11823-11828.
24. Keir M E, et al. Tissue expression of PD-L1 mediates peripheral T cell tolerance. J Exp Med. 2006; 203:883-895.
25. Eppihimer M J, et al. Expression and regulation of the PD-L1 immunoinhibitory molecule on microvascular endothelial cells. Microcirculation. 2002; 9:133-145.
26. Mohan J F, et al. Imaging the emergence and natural progression of spontaneous autoimmune diabetes. Proc Natl Acad Sci USA. 2017; 114:E7776-E7785.
27. Levisetti M G, Suri A, Frederick K, Unanue E R. Absence of lymph nodes in NOD mice treated with lymphotoxin-beta receptor immunoglobulin protects from diabetes. Diabetes. 2004; 53:3115-3119.
28. Gagnerault M-C, Luan J J, Lotton C, Lepault F. Pancreatic lymph nodes are required for priming of beta cell reactive T cells in NOD mice. J Exp Med. 2002; 196:369-377.
29. Wan X, Thomas J W, Unanue E R. Class-switched anti-insulin antibodies originate from unconventional antigen presentation in multiple lymphoid sites. J Exp Med. 2016; 213:967-978.
30. Murayama T, et al. Intraperitoneal administration of anti-c-fms monoclonal antibody prevents initial events of atherogenesis but does not reduce the size of advanced lesions in apolipoprotein E-deficient mice. Circulation. 1999; 99:1740-1746.
31. Segawa M, et al. Suppression of macrophage functions impairs skeletal muscle regeneration with severe fibrosis. Exp Cell Res. 2008; 314:3232-3244.
32. Lim A K H, et al. Antibody blockade of c-fms suppresses the progression of inflammation and injury in early diabetic nephropathy in obese db/db mice. Diabetologia. 2009; 52:1669-1679.
33. Kubota Y, et al. M-CSF inhibition selectively targets pathological angiogenesis and lymphangiogenesis. J Exp Med. 2009; 206:1089-1102.
34. Huynh D, et al. Colony stimulating factor-1 dependence of paneth cell development in the mouse small intestine. Gastroenterology. 2009; 137:136-144e3.
35. Hashimoto D, et al. Pretransplant CSF-1 therapy expands recipient macrophages and ameliorates GVHD after allogeneic hematopoietic cell transplantation. J Exp Med. 2011; 208:1069-1082.
36. Van Rooijen N, Sanders A. Liposome mediated depletion of macrophages: Mechanism of action, preparation of liposomes and applications. J Immunol Methods. 1994; 174:83-93.
37. Lee K U, Amano K, Yoon J W. Evidence for initial involvement of macrophage in development of insulitis in NOD mice. Diabetes. 1988; 37:989-991.
38. Ihm S H, Lee K U, Yoon J W. Studies on autoimmunity for initiation of beta-cell destruction. VII. Evidence for antigenic changes on beta-cells leading to autoimmune destruction of beta-cells in BB rats. Diabetes. 1991; 40:269-274.
39. Jun H S, Santamaria P, Lim H W, Zhang M L, Yoon J W. Absolute requirement of macrophages for the development and activation of beta-cell cytotoxic CD8+ T-cells in T-cell receptor transgenic NOD mice. Diabetes. 1999; 48:34-42.
40. Jun H S, Yoon C S, Zbytnuik L, van Rooijen N, Yoon J W. The role of macrophages in T cell-mediated autoimmune diabetes in nonobese diabetic mice. J Exp Med. 1999; 189:347-358.
41. Calderon B, Suri A, Unanue E R. In CD4+ T-cell-induced diabetes, macrophages are the final effector cells that mediate islet p-cell killing: Studies from an acute model. Am J Pathol. 2006; 169:2137-2147.
42. Calderon B, Suri A, Pan X O, Mills J C, Unanue E R. IFN-gamma-dependent regulatory circuits in immune inflammation highlighted in diabetes. J Immunol. 2008; 181:6964-6974.

### Example 4. Oral Treatment of NOD Mice

Clinical diabetes starts in our colony at about the 20^{th} week of life. A set of mice at 3 weeks of life when the first signal of the autoimmune process is evident in pancreatic islets and at 10 weeks of life when the process has already started were treated. Neither at 3 or 10 weeks is there evidence of beta cell death and mice are euglycemic. Two experiments were done at each time point. Shown are the pooled results of each time point as well as of the combined results of 3 and 10 weeks (Table 1). In the last experiment—7614—a limited number of mice that already had signs of islet inflammation were treated: the four control treated mice (e.g., irrelevant antibody) became diabetic, while so far protection is apparent in the anti-CSF-R antibody treated group, even when the mice were at an advanced stage before treatment commenced. Control refers to mice treated with an irrelevant antibody.

The endpoint for the CSF1R inhibitor treatments was islet infiltration evaluated by flow cytometry. Additionally, the endpoint for the anti-CSF1R antibody treatments was clinical diabetes.

### Methods

Female NOD mice at 4 weeks of age were treated with JNJB:44679292 drug in 2-hydroxypropyl-β-cyclodextrin as carrier. The drug was compounded into the carrier by trituration with a tissue grinder at a concentration of 5 mg/mL drug and 40% 2-hydroxypropyl-β-cyclodextrin in phosphate buffered saline. Mice were administered 0.1 mL of compounded drug orally (0.5 mg drug/dose) daily for 14 days. Control mice received diluted carrier without any drug. At the end of treatment, mice were sacrificed and their islets of Langerhans were examined by flow cytometry to determine their leukocyte composition.

### Results

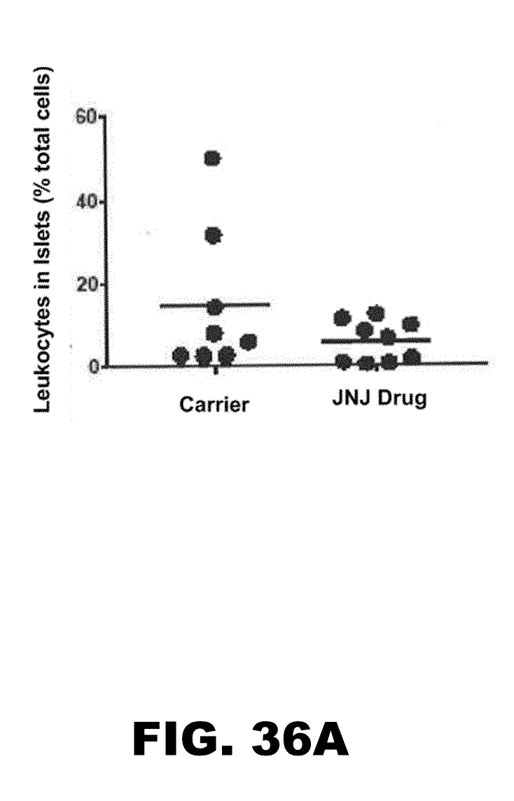

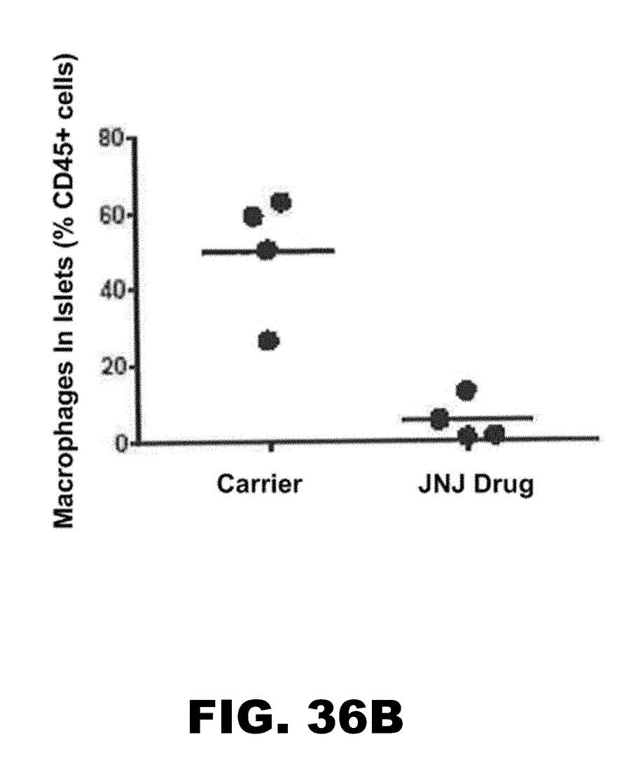

FIG. 36A shows the percent of leukocytes (CD45+ cells) as a function of the total islet cellularity for 8 carrier and 9 drug treated mice. FIG. 36B shows the percent of macrophages as a function of total leukocytes in the islets in 4 carrier and drug treated mice. FIG. 36A shows that leukocytes (mainly T cells) are still found in the islets of 6 week old NOD mice treated with the drug. FIG. 36B shows that the drug depleted all or most of the macrophage in the NOD mice even after T cells had entered the islets.

TABLE 1
Summary of Results

| | Time Started | | |
|---|---|---|---|
| Experiment # | (weeks of life) | Control | AFS tx |
| 1 | 3 | 8/10 | 2/10 |
| 2 | 3 | 8/14 | 1/17 |
| Total | | 16/24:67% | 3/27:11% |
| 1 | 10 | 8/15 | 1/13 |
| 2 | 10 | 7/9 | 2/10 |
| 3 | 10 | 3/3 | 2/5 |
| Total | | 18/27:74% | 5/28:17% |
| Summary | 3-10 | 34/51:67% | 8/55:15% |

All cited references are herein expressly incorporated by reference in their entirety.

Whereas particular embodiments have been described above for purposes of illustration, it will be appreciated by those skilled in the art that numerous variations of the details may be made without departing from the disclosure as described in the appended claims.

What is claimed is:

1. A method of depleting islet macrophages in the pancreas, the method comprising administering to a subject a composition comprising a compound that targets the CSF-1 pathway.

2. The method of claim 1, wherein the compound that disrupts the CSF-1 pathway is a compound that targets CSF-1, IL-34, and/or CSF-1R.

3. The method of claim 1, wherein the compound that disrupts the CSF-1 pathway is an anti-CSF-1 antibody, anti-IL-34 antibody, anti-CSF-1R antibody, or a small molecule tyrosine kinase inhibitor of CSF-1R.

4. The method of claim 1, wherein the compound binds CSF-1R.

5. The method of claim 1, wherein the compound is an anti-CSF-1R antibody.

6. The method of claim 1, wherein the amount of islet macrophages following administration of a composition comprising a compound that disrupts the CSF-1 pathway is at least about 3-fold less than the amount of islet macrophages prior to administration of a composition comprising a compound that disrupts the CSF-1 pathway.

7. A method of selectively depleting islet macrophages in the pancreas relative to tissue macrophages, the method comprising administering to a subject a composition comprising a compound that targets the CSF-1 pathway.

8. The method of claim 7, wherein the compound that disrupts the CSF-1 pathway is a compound that binds CSF-1, IL-34, and/or CSF-1R.

9. The method of claim 7, wherein the compound that disrupts the CSF-1 pathway is an anti-CSF-1 antibody, anti-IL-34 antibody, anti-CSF-1R antibody, or a small molecule tyrosine kinase inhibitor of CSF-1R.

10. The method of claim 7, wherein the compound binds CSF-1R.

11. The method of claim 7, wherein the compound is an anti-CSF-1R antibody.

12. The method of claim 7, wherein the depletion in islet macrophages is greater than the reduction in tissue macrophages following administration of a composition comprising a compound that targets the CSF-1 pathway.

13. The method of claim 12, wherein the depletion in islet macrophages is significantly greater than the reduction in tissue macrophages.

14. The method of claim 12, wherein the depletion in islet macrophages is at least about 3-fold greater than the reduction in tissue macrophages.

15. A method of delaying the onset of autoimmune diabetes, the method comprising depleting islet macrophages in the pancreas of a subject by administering to the subject a composition comprising a compound that disrupts the CSF-1 pathway.

16. The method of claim 15, wherein the compound that disrupts the CSF-1 pathway is a compound that binds CSF-1, IL-34, and/or CSF-1R.

17. The method of claim 15, wherein the compound that disrupts the CSF-1 pathway is an anti-CSF-1 antibody, anti-IL-34 antibody, anti-CSF-1R antibody, or a small molecule tyrosine kinase inhibitor of CSF-1R.

18. The method of claim 15, wherein the compound binds CSF-1R.

19. The method of claim 15, wherein the compound is an anti-CSF-1R antibody.
